(19) United States
(12) Patent Application Publication
(10) Pub. No.: US 2021/0227843 A1
(43) Pub. Date: Jul. 29, 2021

(54) A TEMPERING COMPOSITION FOR TEMPERING GRAIN AND CONTROLLING PATHOGENS IN AND/OR ON SAID GRAIN, AN OXIDIZING COMPOSITION FOR PREPARING SAID TEMPERING COMPOSITION, A USE OF SAID TEMPERING COMPOSITION AND A METHOD OF USE OF SAID TEMPERING COMPOSITION

(71) Applicant: AGRI-NEO INC., Toronto (CA)

(72) Inventors: Fadi DAGHER, Laval (CA); Nicholas DILLON, Toronto (CA); Robert WONG, Toronto (CA); Rebecca HYLTON, Etobicoke (CA); Amir HAMIDI, Etobicoke (CA)

(73) Assignee: AGRI-NEO INC., Toronto, ON (CA)

(21) Appl. No.: 16/757,695

(22) PCT Filed: Oct. 18, 2018
(86) PCT No.: PCT/CA2018/051310

§ 371 Date: Apr. 20, 2020

Related U.S. Application Data
(60) Provisional application No. 62/575,074, filed on Oct. 20, 2017.

(51) Int. Cl. A23B 9/30; A23B 9/26; A61L 2/18

(52) U.S. Cl. CPC A23B 9/30; B02B 1/04; A61L 2/186; A23B 9/26

(57) ABSTRACT

A tempering composition for tempering grain in a tempering step and controlling pathogens susceptible to be present in and/or on said grain during the tempering step of the grain, said tempering step being eventually carried out before subjecting the tempered grain to a milling step; wherein said tempering composition comprises tempering-water and an oxidizing composition comprising at least one oxidizing agent and/or a precursor thereof, and eventually at least one an agriculturally acceptable excipient and/or at least one additive; and wherein the at least one oxidizing agent represents from 0.01 to 50% by weight of the oxidizing composition. A use and a method for tempering grain and controlling pathogens susceptible to be present on grain. An oxidizing composition for preparing the tempering composition. A use of the tempering composition for sanitizing mill systems. A method for sanitizing mill systems.

### CROSS-REFERENCE TO RELATED APPLICATION

The present application is a U.S. national phase of PCT Application No. PCT/CA2018/051310 filed on Oct. 18, 2018, which claims priority to U.S. Provisional Application No. 62/575,074, filed on Oct. 20, 2017. The entire contents of which are incorporated hereby by reference in their entireties.

### BACKGROUND

### Field of the Invention

The present invention relates to a tempering composition for tempering grain and controlling pathogens susceptible to be present in and/or on said grain. The invention also relates to an oxidizing composition for preparing the tempering composition. The invention also relates to a use of a tempering composition for tempering grain in a tempering step and controlling pathogens susceptible to be present in and/or on said grain. The invention also relates to a method for tempering grain and controlling of pathogens susceptible to be present in and/or on said grain. Preferably, pathogens are selected from the group consisting of food pathogens.

### The Statement of the Problem

Recently, the U.S. Food and Drug Administration (FDA) along with the Centers for Disease Control and Prevention (CDC) and state and local public health officials investigated a multi-state outbreak of Shiga toxin-producing Escherichia coli (STEC) O121 and O26 infections. FDA's traceback investigation identified flour from as the source of this outbreak, which led to a voluntary recall in May 2016. FDA tested samples of flour and detected the presence of E. coli O121, which was subsequently confirmed by whole genome sequencing (WGS). In addition, E. coli O26 was also found to be present, prompting an expanded recall (cf. FDA. U.S. Department of Health and Human Services. FDA Investigated Multistate Outbreak of Shiga toxin-producing E. coli Infections Linked to Flour. (2016), which is incorporated herein by reference in its entirety).

Moreover, CDC reported that their outbreak investigation is over. Sixty-three people infected with the outbreak strains of E. coli O121 or O26 were reported from 24 states. Flour has a long shelf life, and bags of flour may be kept in peoples' homes for a long time. Thus, consumers unaware of the recall could continue to use these recalled flour products and potentially get sick.

### Detailed History of the Multi-State Outbreak in the United States

The FDA, CDC, and state and local officials investigated a multi-state outbreak of Shiga toxin-producing Escherichia coli (STEC) O121 and O26 infections. CDC reports that 63 people infected with the outbreak strains of E. coli O121, and O26 were reported from 24 states. Seventeen ill people were hospitalized, and one person developed hemolytic-uremic syndrome. In its investigation, CDC learned that some people who got sick had eaten or handled raw dough.

Despite a voluntary recall of flour products sold in stores nationwide, some products may still be in consumers' pantries. The FDA performed Whole Genome Sequencing (WGS) on E. coli O121 isolates recovered from an open sample of flour belonging to a consumer who was sickened, and it was found to be closely related genetically to the clinical isolates from human illnesses. The flour came from a recalled lot.

Testing by FDA has identified E. coli O121 in open product samples collected from ill people. FDA's WGS analysis of the E. coli O121 isolates from product samples showed that they were closely related genetically to the outbreak strains.

The FDA used WGS to characterize that an E. coli O26 isolated from a returned retail flour is closely related genetically to a clinical isolate that was subsequently added to the outbreak cluster.

### History of the Canadian Outbreak

The food recall warning issued by the Canadian Food Inspection Agency's (CFIA) has been updated to include company which was recalling various brands of flour and flour products due to possible E. coli O121 contamination (cf. CFIA. Updated Food Recall Warning—Various brands of flour and flour products recalled due to E. coli O121. (2017), which is incorporated herein by reference in its entirety). The CFIA advised the Canadian consumers not to consume the recalled products.

On Dec. 29, 2016, PulseNet Canada identified a cluster of six Escherichia coli non-O157 isolates with a matching pulsed-field gel electrophoresis (PFGE) pattern combination that was new to the PulseNet Canada database. The patients resided in three geographically distinct provinces. The Public Health Agency of Canada (PHAC) initiated an investigation with local, provincial, and federal partners to investigate the source of the outbreak (cf. Morton, V., Cheng, J. M., Sharma, D. & Kearney, A. Notes from the Field: An Outbreak of Shiga Toxin-Producing Escherichia coli O121 Infections Associated with Flour—Canada, 2016-2017. MMWR. Morbidity and mortality weekly report 66, 705-706, doi:10.15585/mmwr.mm6626a6 (2017), which is incorporated herein by reference in its entirety).

A case was defined as isolation of E. coli non-O157 with the outbreak PFGE pattern or closely related by whole genome sequencing (WGS) in a Canadian resident or visitor with onset of symptoms of gastroenteritis on or after Nov. 1, 2016. Patients' illness onset dates ranged from November 2016 to April 2017. As of May 23, 2017, a total of 29 cases were identified in six provinces (Alberta, British Columbia, Newfoundland and Labrador, Ontario, Quebec, and Saskatchewan). One additional case was identified in a U.S. resident who traveled to Canada during the exposure period. Patients' ages ranged from 2-79 years (median=23.5 years) and 50% were female. Eight patients were hospitalized, and one developed hemolytic uremic syndrome. Clinical isolates were typed as E. coli O121:H19 (one case was typed as E. coli O121:H undetermined) with Shiga toxin 2-producing genes by in silico toxin testing and had closely related PFGE patterns and WGS.

In March 2017, E. coli O121 with the outbreak PFGE pattern was isolated from an open flour sample from a patient's home and a closed sample collected at a retail store, both of the same brand and production date. The clinical and flour isolates grouped together, with only 0-6 whole genome multi-locus sequence typing allele differences. As a result of these findings, a product recall was issued. Based on possible connections to the recalled lot of flour, market sampling of flour within certain periods was initiated. The investigation led to additional recalls of flour and many secondary products.

22 patients had been asked about flour exposure in the 7 days before illness onset; 16 (73%) reported that the implicated brand of flour was used or probably used in the home during the exposure period. Comparison data on the expected proportion with exposure to this brand of flour were not available. Eleven of these sixteen patients reported they ate or probably ate raw dough during their exposure period.

This was the first Canadian national outbreak of non-O157 Shiga toxing producing E. coli infections identified in Canada and the first Canadian outbreak linked to flour. An open-ended interview approach and flour sampling were used to implicate flour as the source. Because of the recent emergence of E. coli outbreaks linked to flour, public health professionals should consider flour as a possible source in E. coli outbreaks and communicate the risk associated with exposure to flour, raw batter, and dough in public health messaging.

Symptoms Caused by E. coli O121 & O26

People usually get sick from STEC O121 and O26 about 2-8 days (average of 3-4 days) after consuming the contaminated product. Most people develop diarrhea (often bloody) and abdominal cramps. Most people recover within a week (cf. Abbasi, P. et al. Characterization of Shiga-toxin producing E. coli (STEC) and enteropathogenic E. coli (EPEC) using multiplex Real-Time PCR assays for stx1, stx2, eaeA. Iranian journal of microbiology 6, 169-174 (2014); Purwar, S., Roy, S. & Metgud, S. Non-O157:H7 Shiga Toxin Producing Diarrhoeagenic Escherichia coli (STEC) in Southern India: A Tinderbox for Starting Epidemic. Journal of clinical and diagnostic research: JCDR 10, DC11-DC15, doi:10.7860/JCDR/2016/21462.8714 (2016); and Wasilenko, J. L. et al. Detection of Shiga toxin-producing Escherichia coli (STEC) O157:H7, O26, O45, O103, O111, O121, and O145, and Salmonella in retail raw ground beef using the DuPont BAX® system. Frontiers in cellular and infection microbiology 4, 81, doi:10.3389/fcimb.2014.00081 (2014), which are all incorporated herein by reference in their entirety).

Some illnesses last longer and can be more severe, resulting in a type of kidney failure called hemolytic uremic syndrome (HUS). HUS can occur in people of any age, but is most common in young children under 5 years, older adults, and people with weakened immune systems.

Symptoms of HUS can include fever, abdominal pain, pale skin tone, fatigue and irritability, small, unexplained bruises or bleeding from the nose and mouth, and decreased urination.

### Prior Art

Although numerous antimicrobial interventions targeting Escherichia coli O157:H7 have been developed and implemented to decontaminate meat and meat products during the harvesting process, the information on efficacy of these interventions against the so-called Big Six non-O157 Shiga toxin-producing E. coli (STEC) strains is limited (cf. Kalchayanand, N. et al. Evaluation of commonly used antimicrobial interventions for fresh beef inoculated with Shiga toxin-producing Escherichia coli serotypes O26, O45, O103, O111, O121, O145, and O157:H7. Journal of food protection 75, 1207-1212, doi:10.4315/0362-028X.JFP-11-531 (2012), which is incorporated herein by reference in its entirety). In fact, few intervention steps targeting Shiga toxin-producing E. coli and other foodborne pathogens on flour and bran have been described (cf. Lucera, A., Costa, C., Conte, A. & Del Nobile, M. A. Food applications of natural antimicrobial compounds. Frontiers in microbiology 3, 287, doi:10.3389/fmicb.2012.00287 (2012), which is incorporated herein by reference in its entirety). Some acids such as lactic acid citric acids have been described in the literature to have limited effect on wheat in terms of reducing the bacterial load (cf. Sabillón Galeas, L. UNDERSTANDING THE FACTORS AFFECTING MICROBIOLOGICAL QUALITY OF WHEAT MILLED PRODUCTS: FROM WHEAT FIELDS TO MILLING OPERATIONS, University of Nebraska, (2014)). Heat treatment is another option but it affects the functionality of flour (cf. Mann, J., Schiedt, B., Baumann, A., Conde-Petit, B. & Vilgis, T. A. Effect of heat treatment on.wheat dough rheology and wheat protein solubility. Food science and technology international=Ciencia y tecnologia de los alimentos internacional 20, 341-351, doi: 10.1177/1082013213488381 (2014)). Heat treatment causes the formation of gluten aggregates resulting in decreased protein solubility and lower network strength of dough. Rheological data also indicate the formation of starch aggregates and modified interactions between gluten and starch. The effects were more pronounced in heat-treated flours with increased moisture content due to a higher mobility of the molecules.

The Applicant has filed international application WO2015/074144 to control the food pathogens on seeds, nuts and grains. The patent application was related to an effective validated composition to reduce the food pathogen by about 5-log. More particularly, this international application describes a composition of water-soluble ingredients which when solubilized in water and either in the presence of a wetting agent and/or preferably in the presence of at least one agriculturally acceptable solvent, forms a synergistic composition useful for the control of pathogens and/or the prevention of diseases associated with the presence of said pathogens in and/or on seeds.

Also, the Applicant has filed international application WO2016/074099 related to a process of controlling the food pathogens while maintaining the seeds and nuts: raw, organic and viable. More particularly, this international application describes a method for sanitizing edible seeds comprising the steps of providing a sanitizing composition comprising water, at least one biocidal agent and at least one alcohol in an amount (a) effective to suppress the release of mucilage from mucilaginous seeds or (b) providing at least 15% by volume of the composition; applying the sanitizing composition to the seeds; and, drying the seeds.

To the best of Applicant's knowledge, no intervention has targeted specifically the reduction of pathogens, preferably food pathogens, on grain, such as wheat, rice or corn, during the tampering-hold step and/or wheat milling into flour and bran step and/or packaging and storage step while maintaining the moisture content, sensory, nutritional values, physical characteristics and functionality intact and unaltered.

Up to now, oxidizer-containing compositions used for controlling pathogens and/or preventing diseases associated with the presence of said pathogens in and/or on seeds, were related to natural processes inherent to the life of seeds (conservation, growing, etc.).

Also, taking into consideration that grain such as wheat and corn, have special processes in terms of tempering and milling (cf. Zinn, R. A., Alvarez, E. G., Montano, M. F., Plascencia, A. & Ramirez, J. E. Influence of tempering on the feeding value of rolled corn in finishing diets for feedlot cattle. Journal of animal science 76, 2239-2246 (1998); and Haros, M., Rosell, C. M. & Benedito, C. Improvement of flour quality through carbohydrases treatment during wheat tempering. Journal of agricultural and food chemistry 50, 4126-4130 (2002), which are incorporated herein by reference in their entirety), the Applicant has now discovered that a tempering composition obtained by adding in water, at least one oxidizing agent comprising at least one oxidizer and/or a precursor thereof is surprisingly efficient to prevent altering pH, sensory, nutritional values, physical characteristics and functionality of grain such as wheat and flour, and to increase the shelf life of grain (such as wheat) or grain by-products (such as bran), by controlling pathogens such as pathogenic bacteria, yeast and mold.

### SUMMARY OF THE INVENTION

More particularly, the Applicant proposes a novel tempering composition that is applied during the water-addition in the tempering phase aiming at increasing the moisture content of grain to a desired percentage. This novel tempering composition is obtained by mixing an oxidizing composition comprising at least one oxidizing agent and/or a precursor of the at least one oxidizing agent, with tempering-water, and used in the tempering phase. Preferably, a hold-time up to 24 hours maximizes the efficiency of the novel tempering composition in terms of controlling pathogens such as pathogen bacteria, yeast and mold as well as food pathogens such as E. coli spp., Salmonella spp., Listeria spp., etc.

Also, the Applicant proposes a novel oxidizing composition for preparing the novel tempering composition; a new use of the tempering composition for tempering grain in a tempering step and controlling food pathogens susceptible to be present in and/or on said grain; and a novel method for tempering grains and controlling of food pathogens susceptible to be present in and/or on said grain.

Also, since organic food is widely assumed to have a better nutritional quality than conventional food, this novel tempering composition is preferably composed of organic materials and thus can be used on organic grain (such as wheat) and in mills certified as organic (cf. Mazzoncini, M., Antichi, D., Silvestri, N., Ciantelli, G. & Sgherri, C. Organically vs conventionally grown winter wheat: effects on grain yield, technological quality, and on phenolic composition and antioxidant properties of bran and refined flour. Food chemistry 175, 445-451, doi:10.1016/j.foodchem.2014.11.138 (2015), which is incorporated herein by reference in its entirety).

An embodiment of the invention relates to a tempering composition for tempering grain in a tempering step and controlling pathogens, preferably food pathogens, susceptible to be present in and/or on said grain during the tempering step of the grain, said tempering step being eventually carried out before subjecting the tempered grain to a milling step;

wherein said tempering composition comprises tempering-water and an oxidizing composition comprising at least one oxidizing agent and/or a precursor thereof, and eventually at least one an agriculturally acceptable excipient and/or at least one additive; and
wherein the at least one oxidizing agent represents from 0.01 to 50% by weight of the oxidizing composition, preferably from 0.2 to 50% by weight of the oxidizing composition or 0.01 to 10% by weight of the oxidizing composition. Preferably, the control of the amount of pathogens upstream the milling step further allows to control the amount of pathogens in by-products resulting from the milling step and/or control the amount of pathogens in the milling systems.

Another embodiment of the invention relates to the tempering composition defined hereinabove, wherein the oxidizing composition is admixed with the tempering-water in a weight ratio “oxidizing composition:tempering-water” varying from 4:16 to 2:78.

Another embodiment of the invention relates to the tempering composition defined hereinabove, wherein the oxidizing composition is admixed with the tempering-water in a weight ratio “oxidizing composition/tempering-water” varying from 3:16 to 2:78.

Another embodiment of the invention relates to the tempering composition defined hereinabove, wherein the oxidizing composition, the tempering-water and any optional agriculturally acceptable excipient(s) (e.g. good grade excipient(s) such as water and/or a food-grade alcohols, preferably a C₁-C₆ alcohols and more preferably ethanol) and/or or any additive(s) (e.g. acids, stabilizers preferably stabilizers for oxidizers, chelating agents, chlorine dioxide generating agents, etc.), can be mixed together by any appropriate mixing means (in a batch process or a continuous process). Preferably, additive(s) may be selected from the group consisting of acids, stabilizers, more preferably stabilizers for oxidizers, and chelating agents. Preferably, said appropriate mixing means comprise an agitator positioned in a reservoir (batch process) or comprise a combination of pumps (continuous process). Mixing means being well known, they do not need to be further explained.

Another embodiment of the invention relates to the tempering composition defined hereinabove, wherein the tempering composition is applied to the grain at a rate varying from 20 to 80 liters of the tempering composition per ton of grain.

Another embodiment of the invention relates to the tempering composition defined hereinabove, wherein the tempering composition is suitable for contacting the grain for a period varying from 2 to 48 hours, preferably from 2 hours to 30 hours, an more preferably about 16 hours.

Another embodiment of the invention relates to the tempering composition defined hereinabove, wherein said tempering composition is adapted to be applied or contacted with grain by any appropriate means. Preferably, the tempering composition may be applied or contacted with grain by pumping, fumigating, spraying, misting or vaporizing said tempering composition on the grain.

Another embodiment of the invention relates to the tempering composition defined hereinabove, wherein a flow of the tempering composition is to be sprayed on a flow of the grain, and then the mixture of the grain and the tempering composition is to be held in a tempering tank for a period varying from 2 to 48 hours.

Another embodiment of the invention relates to the tempering composition defined hereinabove, wherein the oxidizing composition comprises the at least one oxidizing agent in liquid form or solid form, or the precursor thereof in liquid or solid form.

Another embodiment of the invention relates to the tempering composition defined hereinabove, wherein the agriculturally acceptable excipient can be water and/or at least one food-grade excipient. Preferably, the at least one food-grade excipient is selected from the group consisting of food-grade alcohols, more preferably C₁-C₆ alcohols and much more preferably ethanol, and the at least one additive may comprise (preferably consist of) at least one acid, at least one stabilizing agent, preferably at least one stabilizing agent for oxidizers, at least one chelating agent, and/or at least one chlorine dioxide generating agent, etc. More preferably, the at least one additive is selected from the group consisting of acids, stabilizers, more preferably stabilizers for oxidizers, and chelating agents.

Another embodiment of the invention relates to the tempering composition defined hereinabove, wherein the at least one oxidizing agent comprises:
a) liquid peracid and/or in-situ generated peracid from precursors (e.g. powder precursors);
b) liquid hydrogen peroxide and/or hydrogen peroxide released from a hydrogen peroxide precursor, and/or
c) other liquid oxidizers and/or powder oxidizers generating iodine, chlorine, bromine and/or chlorine dioxide.

Another embodiment of the invention relates to the tempering composition defined hereinabove, wherein the powder precursors of the in-situ generated peracetic acid comprises:
a) a powdered hydrogen peroxide precursor,
b) optionally a pH adjusting agent, and
c) an acetylating agent.

Another embodiment of the invention relates to the tempering composition defined hereinabove, wherein the at least one oxidizing agent comprises:
a) a liquid peracetic acid and/or in-situ generated peracetic acid (e.g. in-situ generated peracetic acid obtained from powder precursors); and/or
b) optionally a liquid hydrogen peroxide and/or hydrogen peroxide released from a hydrogen peroxide precursor (e.g. a powdered hydrogen peroxide precursor);
c) water; and
d) optionally at least one additive and or at least one agriculturally acceptable excipient.

Another embodiment of the invention relates to the tempering composition defined hereinabove, wherein the at least one oxidizing agent comprises:
a) liquid peracetic acid and/or in-situ generated peracetic acid from powder precursors; and
b) liquid hydrogen peroxide and/or hydrogen peroxide released from a powdered hydrogen peroxide precursor.

Another embodiment of the invention relates to the tempering composition defined hereinabove, wherein the at least one oxidizing agent is an oxidizing composition comprising liquid peracetic acid obtained from the reaction of acetic acid with hydrogen peroxide.

The tempering composition, wherein the oxidizing composition comprises
peracetic acid in an amount varying from 2 to 20 weight percent of the oxidizing composition,
water in an amount varying from 80 to 98 weight percent of the oxidizing composition; and
optionally residual amounts of acetic acid and hydrogen peroxide.

Another embodiment of the invention relates to the tempering composition defined hereinabove, wherein the at least one oxidizing agent is an oxidizing composition obtained by admixture of an aqueous solution of peracetic acid obtained from the reaction of acetic acid with hydrogen peroxide; with an aqueous solution of hydrogen peroxide, said oxidizing composition comprising:
peracetic acid in an amount varying from 0.1% to 5% by weight of the oxidizing composition,
hydrogen peroxide in an amount varying from 0.1% to 20% by weight of the oxidizing composition, and
water in an amount varying from 75% to 99.8% by weight, of the oxidizing composition.

Another embodiment of the invention relates to the tempering composition defined hereinabove, wherein the at least one oxidizing agent is liquid hydrogen peroxide.

Another embodiment of the invention relates to the tempering composition defined hereinabove, wherein the liquid hydrogen peroxide of the oxidizing composition is a mixture of hydrogen peroxide and at least one stabilizing agent or hydrogen peroxide as a standalone, the hydrogen peroxide representing from 5 to 50% of the mixture of the oxidizing composition.

Another embodiment of the invention relates to the tempering composition defined hereinabove, wherein the at least one stabilizing agent and/or at least one chelating agent may be ethylenediamine tetraacetic acid (EDTA), nitrilotriacetic Acid (NTA), diethylene triamine pentaacetic acid (DTPA), 1-hydroxyethane(1,1-diylbiphosphonic acid) (HEDP), nitrilotris(methylenephosphonic acid) (NTMP), dipicolinic acid, diethylene triamine pentakis(methylenephosphonic acid) (DTPMP), 1,2-diaminoethanetetrakis (methylenephosphonic acid) (EDTMP), sodium salt of 1,2-diaminoethane tetrakis(methylenephosphonic acid), potassium salt of 1,2-diaminoethane tetrakis(methylenephosphonic acid), ammonium salt of 1,2-diaminoethane tetrakis(methylenephosphonic acid), amino trimethylene phosphonic acid (ATMP), ethylene diamine tetra (methylene phosphonic acid) (EDTMPA Solid), phosphonobutane tricarboxylic acid (PBTCA), polyhydric alcohol phosphate ester (PAPE), 2-hydroxyphosphonocarboxylic acid (HPAA), hexamethylenediamine tetra(methylenephosphonic acid) (HMDTMPA), 1-hydroxyethylidene-1,1-diphosphonic acid (e.g. DEQUEST 2010™), citric acid, bio-succinic acid, or any mixture thereof.

Another embodiment of the invention relates to the tempering composition defined hereinabove, wherein the at least one additive may be sulphuric acid, phosphoric acid or bio-succinic acid, preferably the at least one additive may be sulphuric acid 96%.

Another embodiment of the invention relates to the tempering composition defined hereinabove, wherein the at least one agriculturally acceptable excipient may be water (e.g. distilled water), at least one agriculturally acceptable alcohol or a mixture thereof. More particularly, the at least one agriculturally acceptable alcohol is a food-grade alcohol which is listed in FDA's CFR 21 as Generally Regarded as Safe (GRAS) for use in food (section 184.1293), preferably ethanol, propylene glycol or isopropanol, and more preferably a food-graded ethanol.

Another embodiment of the invention relates to the tempering composition defined hereinabove, wherein the powdered hydrogen peroxide precursor of the oxidizing composition is a persalt.

Another embodiment of the invention relates to the tempering composition defined hereinabove, wherein the persalt is selected from the group consisting of sodium perborate, sodium percarbonate, ammonium percarbonate, sodium peroxyhydrate, calcium peroxide, sodium peroxide, sodium perborate monohydrate, sodium perborate tetrahydrate, sodium persulfate, potassium monopersulfate, perphosphate, magnesium peroxide, zinc peroxide, urea hydrogen peroxide, perhydrate of urea, thiourea dioxide, and mixtures thereof.

Another embodiment of the invention relates to the tempering composition defined hereinabove, wherein the persalt is sodium percarbonate.

Another embodiment of the invention relates to the tempering composition defined hereinabove, wherein the grain may be selected from the group consisting of any kinds of grains, and preferably the grain is wheat, rice or corn.

Another embodiment of the invention relates to the tempering composition defined hereinabove, wherein the grain is wheat, wherein the tempering composition in addition of tempering wheat is:
1) to facilitate the milling, and/or
2) for the control of food pathogens selected from the group consisting of Shiga toxin-producing E. coli strains,
   wherein the at least one oxidizing agent is present in the oxidizing composition at a concentration varying from 0.2 to 50% by weight of the oxidizing composition, and
   wherein said tempering composition preserves functionality and sensory of wheat and/or by-products resulting from the milling step, preferably by-products selected from the group consisting of bran and flour.

Another embodiment of the invention relates to the tempering composition defined hereinabove,

wherein the grain is wheat,
wherein the tempering composition in addition of tempering wheat is for the control of the food pathogens selected from the group consisting of Shiga toxin-producing E. coli O157 strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the tempering composition defined hereinabove,

wherein the grain is wheat,
wherein the tempering composition in addition of tempering wheat is for the control of food pathogens selected from the group consisting of Shiga toxin-producing E. coli O121 strains,
wherein the at least one oxidizing agent is present in the oxidizing composition at a concentration varying 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the tempering composition defined hereinabove,

wherein the grain is wheat,
wherein the tempering composition in addition of tempering wheat is for the control of the food pathogens selected from the group consisting of Salmonella strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the tempering composition defined hereinabove,

wherein the grain is wheat,
wherein the tempering composition in addition of tempering wheat is for the control of the food pathogens selected from the group consisting of Listeria strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the tempering composition defined hereinabove,

wherein the tempering composition in addition of tempering grain is for the control of the food pathogens selected from the group consisting of Shiga toxin-producing E. coli spp, Salmonella spp., and Listeria spp.;
wherein the oxidizing agent is present in the oxidizing composition at a concentration varying from 0.2 to 50% by weight of the oxidizing composition, and wherein said tempering composition does not alter functionality and sensory of grain and/or by-products resulting from the milling step.

Another embodiment of the invention relates to the tempering composition defined hereinabove, wherein the grain is corn.

Another embodiment of the invention relates to the tempering composition defined hereinabove, wherein the at least one oxidizing agent is present in the oxidizing composition at a concentration varying from 0.2 to 50% by weight of the oxidizing composition, and

wherein said tempering composition extends the shelf life of by-products resulting from the milling step, preferably by-products selected from the group consisting of flour and bran but does not alter the functionality and sensory of flour and bran.

Another embodiment of the invention relates to the tempering composition defined hereinabove,

wherein the tempering composition in addition of tempering grain is for the control of the food pathogens selected from the group consisting of aerobic bacteria, yeast and mold,
wherein the at least one oxidizing agent is present in the oxidizing composition at a concentration varying from 0.2 to 50% by weight of the oxidizing composition, and
wherein said tempering composition extends the shelf life of by-products resulting from the milling step, preferably by-products selected from the group consisting of flour and bran, and preserves the functionality and sensory of flour and bran.

Another embodiment of the invention relates to the tempering composition defined hereinabove,

wherein the grain is wheat,
wherein the tempering composition in addition of tempering wheat is for the control of pathogens, preferably food pathogens, and more preferably food pathogens selected from the group consisting of aerobic bacteria, yeast and mold,
wherein the at least one oxidizing agent is present in the oxidizing composition at a concentration varying from 0.2 to 50% by weight of the oxidizing composition, and
wherein said tempering composition prevents the wheat to be bleached or discolored, or preserving the viability of the wheat.

Another embodiment of the invention relates to the tempering composition defined hereinabove,

wherein the grain is wheat,
wherein the tempering composition in addition of tempering wheat is for the control of pathogens, preferably food pathogens, and more preferably food pathogens selected from the group consisting of aerobic bacteria, yeast and mold,
wherein the at least one oxidizing agent is present in the oxidizing composition at a concentration varying from 0.2 to 50% by weight of the oxidizing composition, and leaving the grain free of residues.

Another embodiment of the invention relates to the tempering composition defined hereinabove, wherein residues are selected from the group consisting of peracetic acid, hydrogen peroxide and acetic acid.

Another embodiment of the invention relates to the tempering composition defined hereinabove, wherein the oxidizing composition is applied at a rate of 0.5 to 5 liters of the oxidizing composition per ton of grain, preferably 2 to 4 liters of the oxidizing composition per ton of grain or 3 to 4 liters of the oxidizing composition per ton of grain.

Another embodiment of the invention relates to the tempering composition defined hereinabove, wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

Another embodiment of the invention relates to the tempering composition defined hereinabove, wherein the food pathogens are selected from the group consisting of E. coli O26, E. coli O111, E. coli O103, E. coli O121, E. coli O45, and E. coli O145.

Another embodiment of the invention relates to the tempering composition defined hereinabove, wherein the food pathogens are selected from the group consisting of STEC O157 and non-O157 E. coli.

Another embodiment of the invention relates to the tempering composition defined hereinabove, wherein the food pathogens are selected from the group consisting of E. coli O111, E. coli O26, E. coli O45, E. coli O145, E. coli O104, E. coli O6, E. coli O51, E. coli O103, E. coli O27, and E. coli O84.

Another embodiment of the invention relates to the tempering composition defined hereinabove, useful for the control of E. coli surrogates such as non-pathogenic E. coli cocktail composed of ATTC strains (BAA-1427, BAA-1428, BAA-1429, BAA-1430 and BBA-1431) or Enterococcus faeciurn NRRL B-2354.

Another embodiment of the invention relates to the tempering composition defined hereinabove, wherein the food pathogens is Salmonella spp.

Another embodiment of the invention relates to the tempering composition defined hereinabove, wherein the food pathogens are selected from the group consisting of Salmonella typhimurium, Salmonella choleraesuis and Salmonella enteritidis.

Another embodiment of the invention relates to the tempering composition defined hereinabove, wherein the food pathogens is Listeria spp.

Another embodiment of the invention relates to the tempering composition defined hereinabove, wherein the food pathogens are selected from the group consisting of Listeria monocytogenes, Listeria seeligeri, Listeria ivanovii, Listeria welshimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia and Listeria booriae.

Another embodiment of the invention relates to the tempering composition defined hereinabove, wherein the food pathogens are selected from the group consisting of Salmonella spp, Listeria spp. and E. coli spp.

Another embodiment of the invention relates to the tempering composition defined hereinabove, wherein the food pathogens are selected from the group consisting of Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

Another embodiment of the invention relates to the tempering composition defined hereinabove, wherein during the subsequent milling step the tempering composition further cleans and sanitizes mill systems to thus prevent cross-contamination and reduce the load of microorganisms of said mill systems.

Another embodiment of the invention relates to the tempering composition defined hereinabove, which can be directly used for sanitizing mill systems. In such a case, the mill systems are first manually cleaned to remove dust, residues, biofilms, bacteria, yeast and mold, and then the tempering composition is sprayed or fogged on mill systems to reduce/control the pathogens count. Preferably, pathogens may be bacteria, yeast and mold), and the mill systems may comprise bins, pipings, milling equipments, etc.

Another embodiment of the invention relates to a use of a tempering composition for tempering grain in a tempering step and controlling pathogens, preferably food pathogens, susceptible to be present in and/or on said grain during the tempering step, said tempering step comprising a step of contacting the tempering composition with the grain to thereby obtain a tempered grain, said tempering step being eventually carried out before subjecting the tempered grain to a milling step;

wherein said tempering composition comprises tempering-water and an oxidizing composition comprising at least one oxidizing agent and/or a precursor thereof, and eventually at least one an agriculturally acceptable excipient and/or at least one additive; and
wherein the at least one oxidizing agent represents from 0.01 to 50% by weight of the oxidizing composition, preferably from 0.2 to 50% by weight of the oxidizing composition or 0.01 to 10% by weight of the oxidizing composition. Preferably, the control of the amount of pathogens upstream the milling step further allows to control the amount of pathogens in by-products resulting from the milling step and/or control the amount of pathogens in the milling systems.

Another embodiment of the invention relates to the use defined hereinabove, wherein the oxidizing composition is admixed with the tempering-water in a weight ratio “oxidizing composition:tempering-water” varying from 4:16 to 2:78.

Another embodiment of the invention relates to the use defined hereinabove, wherein the oxidizing composition is admixed with the tempering-water in a weight ratio “oxidizing composition:tempering-water” varying from 3:17 to 2:78.

Another embodiment of the invention relates to the use defined hereinabove, wherein the oxidizing composition, the tempering-water and any optional agriculturally acceptable excipient(s) (e.g. good grade excipient(s) such as water and/or food-grade alcohols, preferably C₁-C₆ alcohols, more preferably ethanol) and/or additive(s) (e.g. acids, stabilizers, preferably stabilizers for oxidizers, chelating agents, chlorine dioxide generating agents, etc.), can be mixed together by any appropriate mixing means. Preferably, additive(s) may be selected from the group consisting of acids, stabilizers, more preferably stabilizers for oxidizers, and chelating agents. Preferably, said appropriate mixing means (in a batch process or a continuous process). Preferably, said appropriate mixing means comprise an agitator positioned in a reservoir (batch process) or comprise a combination of pumps (continuous process). Mixing means being well known, they do not need to be further explained.

Another embodiment of the invention relates to the use defined hereinabove, wherein the tempering composition is applied to the grain at a rate varying from 20 to 80 liters of the tempering composition per ton of grain.

Another embodiment of the invention relates to the use defined hereinabove, wherein the tempering composition contacts the grain for a period varying from 2 hours to 48 hours, preferably from 2 to 30 hours, more preferably about 16 hours.

Another embodiment of the invention relates to the use defined hereinabove, wherein the tempering composition defined hereinabove, is applied or contacted with grain by any appropriate means. Preferably, the tempering composition may be applied by pumping, fumigating, spraying, misting or vaporizing said tempering composition on the grain.

Another embodiment of the invention relates to the use defined hereinabove, wherein a flow of the tempering composition is sprayed on a flow of the grain, and then the mixture of the grain and the tempering composition is held in a tempering tank for a period varying from 2 to 48 hours.

Another embodiment of the invention relates to the use defined hereinabove, wherein said oxidizing composition comprises the at least one oxidizing agent in liquid form or solid form, or a precursor thereof in liquid or solid form.

Another embodiment of the invention relates to the use defined hereinabove, wherein the agriculturally acceptable excipient can be water, and/or at least one food-grade excipient. Preferably the at least one food-grade excipient is selected from the group consisting of food-grade alcohols, more preferably C₁-C₆ alcohols and much more preferably ethanol, and the at least one additive may comprise (preferably consist of) at least one acid, at least one stabilizing agent, preferably at least one stabilizing agent for oxidizers, at least one chelating agent, and/or at least one chlorine dioxide generating agent, etc. More preferably, the at least one additive is selected from the group consisting of acids, stabilizers, more preferably stabilizers for oxidizers, and chelating agents.

Another embodiment of the invention relates to the use defined hereinabove, wherein the at least one oxidizing agent comprises:
a) liquid peracid and/or in-situ generated peracid from precursors (e.g. powder precursors);
b) liquid hydrogen peroxide and/or hydrogen peroxide released from a hydrogen peroxide precursor, and/or
c) other liquid oxidizers and/or powder oxidizers generating iodine, chlorine, bromine and/or chlorine dioxide.

Another embodiment of the invention relates to the use defined hereinabove, wherein the powder precursors of the in-situ generated peracetic acid comprises:
a) a powdered hydrogen peroxide precursor,
b) optionally a pH adjusting agent, and
c) an acetylating agent.

Another embodiment of the invention relates to the use defined hereinabove, wherein the at least one oxidizing agent comprises:
a) a liquid peracetic acid and/or a in-situ generated peracetic acid (e.g. in situ peracetic acid obtained from powder precursors); and/or
b) optionally a liquid hydrogen peroxide and/or hydrogen peroxide released from a hydrogen peroxide precursor (e.g. a powdered hydrogen peroxide precursor);
c) water; and
d) optionally at lest one additive and/or at least one agriculturally acceptable excipient.

Another embodiment of the invention relates to the use defined hereinabove, wherein the at least one oxidizing agent comprises:
a) liquid peracetic acid and/or in-situ generated peracetic acid from powder precursors; and
b) liquid hydrogen peroxide and/or hydrogen peroxide released from a powdered hydrogen peroxide precursor.

Another embodiment of the invention relates to the use defined hereinabove, wherein the at least one oxidizing agent is an oxidizing composition comprising liquid peracetic acid obtained from the reaction of acetic acid with hydrogen peroxide.

Another embodiment of the invention relates to the use defined hereinabove, wherein the oxidizing composition comprises
peracetic acid in an amount varying from 2 to 20 weight percent of the oxidizing composition,
water in an amount varying from 80 to 98 weight percent of the oxidizing composition; and
optionally residual amounts of acetic acid and hydrogen peroxide.

Another embodiment of the invention relates to the use defined hereinabove, wherein the at least one oxidizing agent is an oxidizing composition obtained by admixture of an aqueous solution of peracetic acid obtained from the reaction of acetic acid with hydrogen peroxide; with an aqueous solution of hydrogen peroxide, said oxidizing composition comprising:
peracetic acid in an amount varying from 0.1% to 5% by weight of the oxidizing composition,
hydrogen peroxide in an amount varying from 0.1% to 20% by weight percent of the oxidizing composition, and
water in an amount varying from 75% to 99.8% by weight of the oxidizing composition.

Another embodiment of the invention relates to the use defined hereinabove, wherein the at least one oxidizing agent is liquid hydrogen peroxide.

Another embodiment of the invention relates to the use defined hereinabove, wherein the liquid hydrogen peroxide of the oxidizing composition is a mixture of hydrogen peroxide and at least one stabilizing agent or hydrogen peroxide as a standalone, the hydrogen peroxide representing from 5 to 50% of the mixture of the oxidizing composition.

Another embodiment of the invention relates to the use defined hereinabove, wherein the at least one stabilizing agent and/or at least one chelating agent may be ethylenediamine tetraacetic acid (EDTA), nitrilotriacetic Acid (NTA), diethylene triamine pentaacetic acid (DTPA), 1-hydroxyethane(1,1-diylbiphosphonic acid) (HEDP), nitrilotris(methylenephosphonic acid) (NTMP), dipicolinic acid, diethylene triamine pentakis(methylenephosphonic acid) (DTPMP), 1,2-diaminoethanetetrakis (methylenephosphonic acid) (EDTMP), sodium salt of 1,2-diaminoethane tetrakis(methylenephosphonic acid), potassium salt of 1,2-diaminoethane tetrakis(methylenephosphonic acid), ammonium salt of 1,2-diaminoethane tetrakis(methylenephosphonic acid), amino trimethylene phosphonic acid (ATMP), ethylene diamine tetra (methylene phosphonic acid) (EDTMPA Solid), phosphonobutane tricarboxylic acid (PBTCA), polyhydric alcohol phosphate ester (PAPE), 2-hydroxyphosphonocarboxylic acid (HPAA), hexamethylenediamine tetra(methylenephosphonic acid) (HMDTMPA), 1-hydroxyethylidene-1,1-diphosphonic acid (e.g. DEQUEST 2010™), citric acid, bio-succinic acid, or any mixture thereof].

Another embodiment of the invention relates to the use defined hereinabove, wherein the invention relates to the use defined hereinabove wherein the at least one additive may be sulphuric acid, phosphoric acid or bio-succinic acid, preferably the at least one additive may be sulphuric acid 96%.

According to another embodiment, the invention relates to the use defined hereinabove wherein the at least one agriculturally acceptable excipient may be water (e.g. distilled water), at least one agriculturally acceptable alcohol or a mixture thereof. More particularly, the at least one agriculturally acceptable alcohol is a food-grade alcohol which is listed in FDA's CFR 21 as Generally Regarded as Safe (GRAS) for use in food (section 184.1293), preferably ethanol, propylene glycol or isopropanol, and more preferably a food-graded ethanol.

Another embodiment of the invention relates to the use defined hereinabove, wherein the powdered hydrogen peroxide precursor of the oxidizing composition is a persalt.

Another embodiment of the invention relates to the use defined hereinabove, wherein the persalt is selected from the group consisting of sodium perborate, sodium percarbonate, ammonium percarbonate, sodium peroxyhydrate, calcium peroxide, sodium peroxide, sodium perborate monohydrate, sodium perborate tetrahydrate, sodium persulfate, potassium monopersulfate, perphosphate, magnesium peroxide, zinc peroxide, urea hydrogen peroxide, perhydrate of urea, thiourea dioxide, and mixtures thereof.

Another embodiment of the invention relates to the use defined hereinabove, wherein the persalt is sodium percarbonate.

Another embodiment of the invention relates to the use defined hereinabove, wherein the grain may be selected from the group consisting of any kind of grains, and preferably grain is wheat, rice or corn.

Another embodiment of the invention relates to the use defined hereinabove, wherein the grain is wheat,

wherein the tempering composition in addition of tempering wheat is:

1) to facilitate the milling, and/or
2) for the control of food pathogens selected from the group consisting of Shiga toxin-producing E. coli strains,
   wherein the at least one oxidizing agent is present in the oxidizing composition at a concentration varying from 0.2 to 50% by weight of the oxidizing composition, and
   wherein said tempering composition preserves functionality and sensory of wheat and/or by-products resulting from the milling step, preferably by-products selected from the group consisting of bran and flour.

Another embodiment of the invention relates to the use defined hereinabove,

wherein the grain is wheat,
wherein the tempering composition in addition of tempering wheat is for the control of the food pathogens selected from the group consisting of Shiga toxin-producing E. coli O157 strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the use defined hereinabove,

wherein the grain is wheat,
wherein the tempering composition in addition of tempering wheat is for the control of food pathogens selected from the group consisting of Shiga toxin-producing E. coli O121 strains,
wherein the at least one oxidizing agent is present in the oxidizing composition at a concentration varying 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the use defined hereinabove,

wherein the grain is wheat,
wherein the tempering composition in addition of tempering wheat is for the control of the food pathogens selected from the group consisting of Salmonella strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the use defined hereinabove,

wherein the grain is wheat,
wherein the tempering composition in addition of tempering wheat is for the control of the food pathogens selected from the group consisting of Listeria strains,
wherein the at least one oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the use defined hereinabove,

wherein the tempering composition in addition of tempering grain is for the control of the food pathogens selected from the group consisting of Shiga toxin-producing E. coli spp, Salmonella spp., and Listeria spp.;
wherein the at least one oxidizing agent is present in the oxidizing composition at a concentration varying from 0.2 to 50% by weight of the oxidizing composition, and
wherein said tempering composition does not alter functionality and sensory of grain and/or by-products resulting from the milling step.

Another embodiment of the invention relates to the use defined hereinabove,

wherein the grain is corn.

Another embodiment of the invention relates to the use defined hereinabove,

wherein the at least one oxidizing agent is present in the oxidizing composition at a concentration varying from 0.2 to 50% by weight of the oxidizing composition, and
wherein said tempering composition extends the shelf life of by-products resulting from the milling step, preferably by-products selected from the group consisting of flour and bran but does not alter the functionality and sensory of flour and bran.

Another embodiment of the invention relates to the use defined hereinabove,

wherein the tempering composition in addition of tempering grain is for the control of pathogens, preferably food pathogens and more preferably food pathogens selected from the group consisting of aerobic bacteria, yeast and mold,
wherein the at least one oxidizing agent is present in the oxidizing composition at a concentration varying from 0.2 to 50% by weight of the oxidizing composition, and
wherein said tempering composition extends the shelf life of by-products resulting from the milling step, preferably by-products selected from the group consisting of flour and bran, and preserves the functionality and sensory of flour and bran.

Another embodiment of the invention relates to the use defined hereinabove,

wherein the grain is wheat,
wherein the tempering composition in addition of tempering wheat is for the control of pathogens, preferably food pathogens and more preferably food pathogens selected from the group consisting of aerobic bacteria, yeast and mold,
wherein the at least one oxidizing agent is present in the oxidizing composition at a concentration varying from 0.2 to 50% by weight of the oxidizing composition, and
wherein said tempering composition prevents the wheat to be bleached or discolored, or preserves the viability of the wheat.

Another embodiment of the invention relates to the use defined hereinabove,

wherein the grain is wheat,
wherein the tempering composition in addition of tempering wheat is for the control of pathogens, preferably food pathogens and more preferably food pathogens selected from the group consisting of aerobic bacteria, yeast and mold,
wherein the at least one oxidizing agent is present in the oxidizing composition at a concentration varying from 0.2 to 50% by weight of the oxidizing composition, and
wherein said tempering composition leaves the grain free of residues.

Another embodiment of the invention relates to the use defined hereinabove, wherein residues are selected from the group consisting of peracetic acid, hydrogen peroxide and acetic acid.

Another embodiment of the invention relates to the use defined hereinabove, wherein the oxidizing composition is applied at a rate of 0.5 to 5 liters of the oxidizing composition per ton of grain, preferably 2 to 4 liters of the oxidizing composition per ton of grain or 3 to 4 liters of the oxidizing composition per ton of grain.

Another embodiment of the invention relates to the use defined hereinabove,

wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

Another embodiment of the invention relates to the use defined hereinabove,

wherein the food pathogens are selected from the group consisting of E. coli O26, E. coli O111, E. coli O103, E. coli O121, E. coli O45, and E. coli O145.

Another embodiment of the invention relates to the use defined hereinabove,

wherein the food pathogens are selected from the group consisting of STEC O157 and non-O157 E. coli.

Another embodiment of the invention relates to the use defined hereinabove,

wherein the food pathogens are selected from the group consisting of E. coli O111, E. coli O26, E. coli O45, E. coli O145, E. coli O104, E. coli O6, E. coli O51, E. coli O103, E. coli O27, and E. coli O84.

Another embodiment of the invention relates to the use defined hereinabove,

wherein the food pathogens is Salmonella spp.

Another embodiment of the invention relates to the use defined hereinabove, wherein the food pathogens are selected from the group consisting of Salmonella typhimurium, Salmonella choleraesuis and Salmonella enteritidis.

Another embodiment of the invention relates to the use defined hereinabove, wherein the food pathogens is Listeria spp.

Another embodiment of the invention relates to the use defined hereinabove, wherein the food pathogens are selected from the group consisting of Listeria monocytogenes, Listeria seeligeri, Listeria ivanovii, Listeria welshimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia and Listeria booriae.

Another embodiment of the invention relates to the use defined hereinabove, wherein the food pathogens are selected from the group consisting of Salmonella spp, Listeria spp. and E. coli spp.

Another embodiment of the invention relates to the use defined hereinabove, wherein the food pathogens are selected from the group consisting of Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

Another embodiment of the invention relates to the use defined hereinabove, wherein during the subsequent milling step the tempering composition further cleans and sanitizes mill systems to thus prevent cross-contamination and reduce the load of microorganisms in the milling systems.

Another embodiment of the invention relates to the use of the tempering composition defined hereinabove, for sanitazing mill systems. In such a case, mill systems are first manually cleaned to remove dust, residues, biofilms, bacteria, yeast and mold, and then the tempering composition defined hereinabove is sprayed or fogged on mill systems to reduce/control the pathogens count. Preferably, the pathogens may comprise bacteria, yeast and mold, and the mill systems may comprise bins, pipings, milling equipments, etc.

Another embodiment of the invention relates to a method for tempering grain and for the control of pathogens, preferably food pathogens, susceptible to be present in and/or on said grain during a tempering step of the grain with a tempering composition to thereby obtain a tempered grain, said tempering step being eventually carried out before subjecting the tempered grain to a milling step;

wherein said method comprises a step of contacting the tempering composition with the grain to thereby obtain the tempered grain,
wherein the tempering composition comprises tempering-water and an oxidizing composition comprising at least one oxidizing agent and/or a precursor thereof, and eventually at least one an agriculturally acceptable excipient and/or at least on additive; and
wherein the at least one oxidizing agent represents 0.01 to 50% by weight of the oxidizing composition, preferably from 0.2 to 50% by weight of the oxidizing composition or from 0.01 to 10% by weight of the oxidizing composition. Preferably, the control of the amount of pathogens upstream the milling step further allows to control the amount of pathogens in by-products resulting from the milling step and/or control the amount of pathogens in the milling systems.

Another embodiment of the invention relates to the method defined hereinabove, wherein the oxidizing composition is admixed with the tempering-water in a weight ratio “oxidizing composition:tempering-water” varying from 4:16 to 2:78.

Another embodiment of the invention relates to the method defined hereinabove, wherein the oxidizing composition is admixed with the tempering-water in a weight ratio “oxidizing composition/tempering-water” varying from 3:16 to 2:78.

Another embodiment of the invention relates to the method defined hereinabove, wherein the oxidizing composition, the tempering-water and any optional agriculturally acceptable excipients (e.g. good grade excipient(s) such as water and/or food-grade alcohols, preferably C₁-C₆ alcohols and more preferably ethanol) and/or any additives (e.g. stabilizers, preferably stabilizers for oxidizers, chelating agents, chlorine dioxide generating agents, etc.), can be mixed together by any appropriate mixing means (in a batch process or a continuous process). Preferably, additive(s) may be selected from the group consisting of acids, stabilizers, more preferably stabilizers for oxidizers, and chelating agents. Preferably, said appropriate mixing means comprise an agitator positioned in a reservoir (batch process) or comprise a combination of pumps (continuous process). Mixing means being well known, they do not need to be further explained.

Another embodiment of the invention relates to the method defined hereinabove, wherein the tempering composition is applied to the grain at a rate varying from 20 to 80 liters of the tempering composition per ton of grain.

Another embodiment of the invention relates to the method defined hereinabove, wherein the tempering composition contacts the grain for a period varying from 2 hours to 48 hours, preferably from 2 to 30 hours, and more preferably about 16 hours.

Another embodiment of the invention relates to the method defined hereinabove, wherein the tempering composition is applied or contacted with grain by any appropriate means. Preferably, the tempering composition may be applied or contacted with grain by pumping, fumigating, spraying, misting or vaporizing said tempering composition on the grain.

Another embodiment of the invention relates to the method defined hereinabove, wherein a flow of the tempering composition is sprayed on a flow of the grain, and then the mixture of the grain and the tempering composition is held in a tempering tank for a period varying from 2 to 48 hours.

Another embodiment of the invention relates to the method defined hereinabove, wherein said oxidizing composition comprises the at least one oxidizing agent in liquid form or solid form, or the precursor thereof in liquid or solid form.

Another embodiment of the invention relates to the method defined hereinabove, wherein the agriculturally acceptable excipient can be water and/or at least one food-grade excipient. Preferably, the at least one food-grade excipient is selected from the group consisting of food-grade alcohols, preferably C₁-C₆ alcohols and more preferably ethanol, and the at least one additive may comprise (preferably consist of) at least one acid, at least one stabilizer, preferably stabilizers for oxidizers, at least one chelating agent, and/or at least one chlorine dioxide generating agent, etc. More preferably, the at least one additive is selected from the group consisting of acids, stabilizers, more preferably stabilizers for oxidizers, and chelating agents.

Another embodiment of the invention relates to the method defined hereinabove, wherein the at least one oxidizing agent comprises:
a) liquid peracid and/or in-situ generated peracid from precursors (e.g. powder precursors);
b) liquid hydrogen peroxide and/or hydrogen peroxide released from a hydrogen peroxide precursor, and/or
c) other liquid oxidizers and/or powder oxidizers generating iodine, chlorine, bromine and/or chlorine dioxide.

Another embodiment of the invention relates to the method defined hereinabove, wherein the powder precursors of the in-situ generated peracetic acid comprises:
a) a powdered hydrogen peroxide precursor,
b) optionally a pH adjusting agent, and
c) an acetylating agent.

Another embodiment of the invention relates to the method defined hereinabove, wherein the at least one oxidizing agent comprises:
a) a liquid peracetic acid and/or a in-situ generated peracetic acid (e.g. per acetic acid obtained from powder precursors); and/or
b) optionally a liquid hydrogen peroxide and/or hydrogen peroxide released from a hydrogen peroxide precursor (e.g. a powdered hydrogen peroxide precursor);
c) water; and
d) optionally at least one additive and/or at least one agriculturally acceptable excipient.

Another embodiment of the invention relates to the method defined hereinabove, wherein the oxidizing composition comprises
peracetic acid in an amount varying from 2 to 20 weight percent of the oxidizing composition,
water in an amount varying from 80 to 98 weight percent of the oxidizing composition; and
optionally residual amounts of acetic acid and hydrogen peroxide.

Another embodiment of the invention relates to the method defined hereinabove, wherein the at least one oxidizing agent comprises:
a) liquid peracetic acid and/or in-situ generated peracetic acid from powder precursors; and
b) liquid hydrogen peroxide and/or hydrogen peroxide released from a powdered hydrogen peroxide precursor.

Another embodiment of the invention relates to the method defined hereinabove, wherein the at least one oxidizing agent is an oxidizing composition comprising liquid peracetic acid obtained from the reaction of acetic acid with hydrogen peroxide.

Another embodiment of the invention relates to the method defined hereinabove, wherein the at least one oxidizing agent is an oxidizing composition obtained by admixture of an aqueous solution of peracetic acid obtained from the reaction of acetic acid with hydrogen peroxide; with an aqueous solution of hydrogen peroxide, said oxidizing composition comprising:
peracetic acid in an amount varying from 0.1% to 5% by weight of the oxidizing composition,
hydrogen peroxide in an amount varying from 0.1 to 20% by weight of the oxidizing composition, and
water in an amount varying from 75% to 99.8% by weight of the oxidizing composition.

Another embodiment of the invention relates to the method defined hereinabove, wherein the at least one oxidizing agent is liquid hydrogen peroxide.

Another embodiment of the invention relates to the method defined hereinabove, wherein the liquid hydrogen peroxide of the oxidizing composition is a mixture of hydrogen peroxide and at least one stabilizing agent or hydrogen peroxide as a standalone, the hydrogen peroxide representing from 5 to 50% by weight of the mixture of the oxidizing composition.

Another embodiment of the invention relates to the method defined hereinabove, wherein the at least one stabilizing agent and/or at least one chelating agent may be ethylenediamine tetraacetic acid (EDTA), nitrilotriacetic Acid (NTA), diethylene triamine pentaacetic acid (DTPA), 1-hydroxyethane(1,1-diylbiphosphonic acid) (HEDP), nitrilotris(methylenephosphonic acid) (NTMP), dipicolinic acid, diethylene triamine pentakis(methylenephosphonic acid) (DTPMP), 1,2-diaminoethanetetrakis (methylenephosphonic acid) (EDTMP), sodium salt of 1,2-diaminoethane tetrakis(methylenephosphonic acid), potassium salt of 1,2-diaminoethane tetrakis(methylenephosphonic acid), ammonium salt of 1,2-diaminoethane tetrakis(methylenephosphonic acid), amino trimethylene phosphonic acid (ATMP), ethylene diamine tetra (methylene phosphonic acid) (EDTMPA Solid), phosphonobutane tricarboxylic acid (PBTCA), polyhydric alcohol phosphate ester (PAPE), 2-hydroxyphosphonocarboxylic acid (HPAA), hexamethylenediamine tetra(methylenephosphonic acid) (HMDTMPA), 1-hydroxyethylidene-1,1-diphosphonic acid (e.g. DEQUEST 2010™), citric acid, bio-succinic acid, or any mixture thereof.

Another embodiment of the invention relates to the method defined hereinabove, wherein the at least one additive may be sulphuric acid, phosphoric acid or bio-succinic acid, preferably the at least one additive may be sulphuric acid 96%.

Another embodiment of the invention relates to the method defined hereinabove, wherein the at least one agriculturally acceptable excipient may be water (e.g. distilled water), at least one agriculturally acceptable alcohol or a mixture thereof. More particularly, the at least one agriculturally acceptable alcohol is a food-grade alcohol which is listed in FDA's CFR 21 as Generally Regarded as Safe (GRAS) for use in food (section 184.1293), preferably ethanol, propylene glycol or isopropanol, and more preferably a food-graded ethanol.

Another embodiment of the invention relates to the method defined hereinabove, wherein the powdered hydrogen peroxide precursor is a persalt.

Another embodiment of the invention relates to the method defined hereinabove, wherein the persalt is selected from the group consisting of sodium perborate, sodium percarbonate, ammonium percarbonate, sodium peroxyhydrate, calcium peroxide, sodium peroxide, sodium perborate monohydrate, sodium perborate tetrahydrate, sodium persulfate, potassium monopersulfate, perphosphate, magnesium peroxide, zinc peroxide, urea hydrogen peroxide, perhydrate of urea, thiourea dioxide, and mixtures thereof.

Another embodiment of the invention relates to the method defined hereinabove, wherein the persalt is sodium percarbonate.

Another embodiment of the invention relates to the method defined hereinabove, wherein the grain is selected from the group consisting of any kind of grains, and preferably the grain is wheat, rice or corn.

Another embodiment of the invention relates to the method defined hereinabove,

wherein the grain is wheat,
wherein the tempering composition in addition of tempering wheat is
1) to facilitate the milling, and/or
2) for the control of food pathogens selected from the group consisting of Shiga toxin-producing E. coli strains,
   wherein the oxidizing agent is present in the oxidizing composition at a concentration varying from 2 to 50% by weight of the oxidizing composition, and
   wherein said tempering composition preserves functionality and sensory of wheat and/or by-products resulting from the milling step, preferably by-products selected from the group consisting of bran and flour.

Another embodiment of the invention relates to the method defined hereinabove,

wherein the grain is wheat,
wherein the tempering composition in addition of tempering grain is for the control of the food pathogens selected from the group consisting of Shiga toxin-producing E. coli O157 strains,
wherein the oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the method defined hereinabove,

wherein the grain is wheat,
wherein the tempering composition in addition of tempering wheat is for the control of food pathogens selected from the group consisting of Shiga toxin-producing E. coli O121 strains,
wherein the oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the method defined hereinabove,

wherein the grain is wheat,
wherein the tempering composition in addition of tempering wheat is for the control of the food pathogens selected from the group consisting of Salmonella strains,
wherein the oxidizing agent is present in the oxidizing composition at a concentration varying from 0.2 to 50% by weight of the oxidizing composition, and
wherein said tempering composition preserves preserving functionality and sensory of wheat and/or by-products resulting from the milling step, preferably by-products selected from the group consisting of bran and flour.

Another embodiment of the invention relates to the method defined hereinabove,

wherein the grain is wheat,
wherein the tempering composition in addition of tempering wheat is for the control of the food pathogens selected from the group consisting of Listeria strains,
wherein the oxidizing agent is present in the oxidizing composition at a concentration varying from 0.2 to 50% by weight of the oxidizing composition, and
wherein said tempering composition preserves functionality and sensory of wheat and/or by-products resulting from the milling step, preferably by-products selected from the group consisting of bran and flour.

Another embodiment of the invention relates to the method defined hereinabove,

wherein the tempering composition in addition of tempering grain is for the control of the food pathogens selected from the group consisting of Shiga toxin-producing E. coli spp, Salmonella spp. and Listeria spp.;
wherein the oxidizing agent is present in the oxidizing composition at a concentration varying from 0.2 to 50% by weight of the oxidizing composition, and
wherein said tempering composition does not alter functionality and sensory of grain and/or by-products resulting from the milling step.

Another embodiment of the invention relates to the method defined hereinabove, wherein the grain is corn.

Another embodiment of the invention relates to the method defined hereinabove, wherein the at least one oxidizing agent is present in the oxidizing composition at a concentration varying from 0.2 to 50% by weight of the oxidizing composition, and

wherein said tempering composition extends the shelf life of by-products resulting from the milling step and selected from the group consisting of flour and bran and preserves the functionality and sensory of flour and bran.

Another embodiment of the invention relates to the method defined hereinabove,

wherein the tempering composition in addition of tempering grain is for the control of pathogens, preferably food pathogens and more preferably food pathogens selected from the group consisting of aerobic bacteria, yeast and mold,
wherein the oxidizing agent is present in the oxidizing composition at a concentration varying from 0.2 to 50% by weight of the oxidizing composition, and
wherein said tempering composition extends the shelf life of by-products resulting from the milling step and selected from the group consisting of flour and bran and preserves the functionality and sensory of flour and bran.

Another embodiment of the invention relates to the method defined hereinabove,

wherein the grain is wheat,
wherein the tempering composition in addition of tempering wheat is for the control of pathogens, preferably food pathogens and more preferably food pathogens selected from the group consisting of aerobic bacteria, yeast and mold,
wherein the oxidizing agent is present in the oxidizing composition at a concentration varying from 0.2 to 50% by weight of the oxidizing composition, and
wherein said tempering composition prevents the wheat to be bleached, discolored or altered.

Another embodiment of the invention relates to the method defined hereinabove,

wherein the grain is wheat,
wherein the tempering composition in addition of tempering wheat is for the control of pathogens, preferably food pathogens and more preferably food pathogens selected from the group consisting of aerobic bacteria, yeast and mold,
wherein the oxidizing agent is present in the oxidizing composition at a concentration varying from 0.2 to 50% by weight of the oxidizing composition, and
wherein said tempering composition leaves the wheat free of residues.

Another embodiment of the invention relates to the method defined hereinabove, wherein residues are selected from the group consisting of peracetic acid, hydrogen peroxide and acetic acid.

Another embodiment of the invention relates to the method defined hereinabove, wherein the oxidizing composition is applied at a rate of 0.5 to 5 liters of the oxidizing composition per ton of grain, preferably of 2 to 4 liters of the oxidizing composition per ton of grain or 3 to 4 liters of the oxidizing composition per ton of grain.

Another embodiment of the invention relates to the method defined hereinabove, wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

Another embodiment of the invention relates to the method defined hereinabove, wherein the food pathogens are selected from the group consisting of E. coli O26, E. coli O111, E. coli O103, E. coli O121, E. coli O45, and E. coli O145.

Another embodiment of the invention relates to the method defined hereinabove, wherein the food pathogens are selected from the group consisting of STEC O157 and non-O157 E. coli.

Another embodiment of the invention relates to the method defined hereinabove, wherein the food pathogens are selected from the group consisting of E. coli O111; E. coli O26; E. coli O45; E. coli O145, E. coli O104, E. coli O6; E. coli O51; E. coli 0103; E. coli O27; and, E. coli O84.

Another embodiment of the invention relates to the method defined hereinabove, wherein the food pathogens is of Salmonella spp.

Another embodiment of the invention relates to the method defined hereinabove, wherein the food pathogens are selected from the group consisting of Salmonella typhimurium, Salmonella choleraesuis and Salmonella enteritidis.

Another embodiment of the invention relates to the method defined hereinabove, wherein the food pathogens are selected from the group consisting of Listeria spp.

Another embodiment of the invention relates to the method defined hereinabove, wherein the food pathogens are selected from the group consisting of Listeria monocytogenes, Listeria seeligeri, Listeria ivanovii, Listeria weishimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia and Listeria booriae.

Another embodiment of the invention relates to the method defined hereinabove, wherein the food pathogens are selected from the group consisting of Salmonella spp, Listeria spp. and E. coli spp.

Another embodiment of the invention relates to the method defined hereinabove, wherein the food pathogens are selected from the group consisting of Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

Another embodiment of the invention relates to the method defined hereinabove, wherein during the subsequent milling step the tempering composition further cleans and sanitizes mill systems to prevent cross-contamination and reduce the load of microorganisms in the milling systems.

Another embodiment of the invention relates to the method for sanitizing mill systems. In such a case, mill systems are first manually cleaned to remove dust, residues, biofilms, bacteria, yeast and mold, and then the tempering composition is contacted with mill systems. Preferably, the tempering composition is sprayed or fogged on mill systems to reduce/control the pathogens count. Preferably, the pathogens may comprise at least one of bacteria, yeasts and/or molds), and mill systems may comprise at least one of bins, pipings, milling equipment, etc.

Another embodiment of the invention relates to an oxidizing composition for the preparation of a tempering composition useful for tempering grain in a tempering step and controlling food pathogens susceptible to be present in and/or on said grain during the tempering step of the grain, said tempering step being to be eventually carried out before subjecting the tempered grain to a milling step;

wherein said tempering composition comprises tempering-water and an oxidizing composition;
wherein the oxidizing composition comprises at least one oxidizing agent and/or a precursor thereof, and eventually at least one an agriculturally acceptable excipient and/or at least one additive; and
wherein the at least one oxidizing agent represents from 0.01 to 50% by weight of the oxidizing composition. Preferably, the control of the amount of pathogens upstream the milling step further allows to control the amount of pathogens in by-products resulting from the milling step and/or control the amount of pathogens in the milling systems.

Another embodiment of the invention relates to the oxidizing composition as defined hereinabove, wherein the oxidizing composition is adapted to be admixed with the tempering-water in a weight ratio “oxidizing composition:tempering-water” varying from 4:16 to 2:78.

Another embodiment of the invention relates to the oxidizing composition as defined hereinabove, wherein the oxidizing composition is adapted to be admixed with the tempering-water in a weight ratio “oxidizing composition/tempering-water” varying from 3:16 to 2:78.

Another embodiment of the invention relates to the oxidizing composition defined hereinabove, wherein the oxidizing composition, the tempering-water and any optional agriculturally acceptable excipient(s) (e.g. good grade excipient(s) such as water and/or food-grade alcohols, preferably C₁-C₆ alcohols and more preferably ethanol) and/or additive(s) (e.g. stabilizers, preferably stabilizers for oxidizers, chelating agents, chlorine dioxide generating agents, etc.), can be mixed together by any appropriate mixing means (in a batch process or a continuous process). Preferably, additive(s) may be selected from the group consisting of acids, stabilizers, more preferably stabilizers for oxidizers, and chelating agents. Preferably, said appropriate mixing means comprise an agitator positioned in a reservoir (batch process) or comprise a combination of pumps (continuous process). Mixing means being well known, they do not need to be further explained.

Another embodiment of the invention relates to the oxidizing composition as defined hereinabove, wherein the tempering composition is adapted to be applied to the grain at a rate varying from 20 to 80 liters of the tempering composition per ton of grain.

Another embodiment of the invention relates to the oxidizing composition as defined hereinabove, wherein the tempering composition is suitable for contacting the grain for a period varying from 2 to 48 hours.

Another embodiment of the invention relates to the oxidizing composition defined hereinabove, wherein the oxidizing composition is suitable for the preparation of the tempering composition defined hereinabove, said tempering composition being adapted to be applied or contacted with grain by any appropriate means. Preferably, the tempering composition may be applied by pumping, fumigating, spraying, misting or vaporizing said tempering composition on the grain.

Another embodiment of the invention relates to the oxidizing composition defined hereinabove, wherein the oxidizing composition is suitable for the preparation of the tempering composition defined hereinabove, and wherein a flow of the tempering composition is to be sprayed on a flow of the grain, and then the mixture of the grain and the tempering composition is to be held in a tempering tank for a period varying from 2 to 48 hours.

Another embodiment of the invention relates to the oxidizing composition as defined hereinabove, comprising the at least one oxidizing agent in liquid form or solid form, or the precursor thereof in liquid or solid form.

Another embodiment of the invention relates to the oxidizing composition defined hereinabove, wherein the oxidizing composition is suitable for the preparation of the tempering composition defined hereinabove, and wherein the excipient can be water and/or a food-grade excipient selected from the group consisting of C₁-C₆ alcohols, and the at least one additive may comprise (preferably consist of) at least one acid, at least one stabilizing agent, preferably at least one stabilizing agent for oxidizers, at least one chelating agent, and/or at least one chlorine dioxide generating agent, etc. More preferably, the at least one additive is selected from the group consisting of acids, stabilizers, more preferably stabilizers for oxidizers, and chelating agents.

Another embodiment of the invention relates to the oxidizing composition as defined hereinabove, wherein the at least one oxidizing agent comprises:
a) liquid peracid and/or in-situ generated peracid from precursors (e.g. powder precursors);
b) liquid hydrogen peroxide and/or hydrogen peroxide released from a hydrogen peroxide precursor, and/or
c) other liquid oxidizers and/or powder oxidizers generating iodine, chlorine, bromine and/or chlorine dioxide.

Another embodiment of the invention relates to the oxidizing composition defined hereinabove, wherein the powder precursors of the in-situ generated peracetic acid comprises:
a) a powdered hydrogen peroxide precursor,
b) optionally a pH adjusting agent, and
c) an acetylating agent.

Another embodiment of the invention relates to the oxidizing composition as defined hereinabove, wherein the at least one oxidizing agent comprises:
a) a liquid peracetic acid and/or a in-situ generated peracetic acid (e.g. a in situ generated peracetic acid obtained from powder precursors);
b) optionally a liquid hydrogen peroxide and/or hydrogen peroxide released from a hydrogen peroxide precursor (e.g. a powdered hydrogen peroxide precursor);
c) water; and
d) optionally at least one additive and/or at least one agriculturally acceptable excipient.

Another embodiment of the invention relates to the oxidizing composition as defined hereinabove, wherein the at least one oxidizing agent comprises:
a) liquid peracetic acid and/or in-situ generated peracetic acid from powder precursors; and
b) liquid hydrogen peroxide and/or hydrogen peroxide released from a powdered hydrogen peroxide precursor.

Another embodiment of the invention relates to the oxidizing composition as defined hereinabove, wherein the at least one oxidizing agent is liquid peracetic acid obtained from the reaction of acetic acid with hydrogen peroxide.

Another embodiment of the invention relates to the oxidizing composition as defined hereinabove, comprising
peracetic acid in an amount varying from 2 to 20 weight percent of the oxidizing composition,
water in an amount varying from 80 to 98 weight percent of the oxidizing composition; and
optionally residual amounts of acetic acid and hydrogen peroxide.

Another embodiment of the invention relates to the oxidizing composition as defined hereinabove, obtained by admixture of an aqueous solution of peracetic acid obtained from the reaction of acetic acid with hydrogen peroxide; with an aqueous solution of hydrogen peroxide, said oxidizing composition comprising:
peracetic acid in an amount varying from 0.1% to 5% by weight of the oxidizing composition,
hydrogen peroxide in an amount varying from 0.1% to 20% by weight of the oxidizing composition, and
water in an amount varying from 75% to 99.8% by weight of the oxidizing composition.

Another embodiment of the invention relates to the oxidizing composition as defined hereinabove, wherein the at least one oxidizing agent is liquid hydrogen peroxide.

Another embodiment of the invention relates to the oxidizing composition as defined hereinabove, wherein the liquid hydrogen peroxide is a mixture of hydrogen peroxide and at least one stabilizing agent or hydrogen peroxide as a standalone, the hydrogen peroxide representing from 5 to 50% of the mixture of the oxidizing composition.

Another embodiment of the invention relates to the oxidizing composition defined hereinabove, wherein the at least one stabilizing agent and/or at least one chelating agent may be ethylenediamine tetraacetic acid (EDTA), nitrilotriacetic Acid (NTA), diethylene triamine pentaacetic acid (DTPA), 1-hydroxyethane(1,1-diylbiphosphonic acid) (HEDP), nitrilotris(methylenephosphonic acid) (NTMP), dipicolinic acid, diethylene triamine pentakis(methylenephosphonic acid) (DTPMP), 1,2-diaminoethanetetrakis (methylenephosphonic acid) (EDTMP), sodium salt of 1,2-diaminoethane tetrakis(methylenephosphonic acid), potassium salt of 1,2-diaminoethane tetrakis(methylenephosphonic acid), ammonium salt of 1,2-diaminoethane tetrakis(methylenephosphonic acid), amino trimethylene phosphonic acid (ATMP), ethylene diamine tetra (methylene phosphonic acid) (EDTMPA Solid), phosphonobutane tricarboxylic acid (PBTCA), polyhydric alcohol phosphate ester (PAPE), 2-hydroxyphosphonocarboxylic acid (HPAA), hexamethylenediamine tetra(methylenephosphonic acid) (HMDTMPA), 1-hydroxyethylidene-1,1-diphosphonic acid (e.g. DEQUEST 2010™), citric acid, bio-succinic acid, or any mixture thereof.

Another embodiment of the invention relates to the oxidizing composition defined hereinabove, wherein the at least one additive may be sulphuric acid, phosphoric acid or bio-succinic acid, preferably the at least one additive may be sulphuric acid 96%.

Another embodiment of the invention relates to the oxidizing composition defined hereinabove, wherein the at least one agriculturally acceptable excipient may be water (e.g. distilled water), at least one agriculturally acceptable alcohol or a mixture thereof. More particularly, the at least one agriculturally acceptable alcohol is a food-grade alcohol which is listed in FDA's CFR 21 as Generally Regarded as Safe (GRAS) for use in food (section 184.1293), preferably ethanol, propylene glycol or isopropanol, and more preferably a food-graded ethanol.

Another embodiment of the invention relates to the oxidizing composition as defined hereinabove, wherein the powdered hydrogen peroxide precursor of the oxidizing composition is a persalt.

Another embodiment of the invention relates to the oxidizing composition as defined hereinabove, wherein the persalt is selected from the group consisting of sodium perborate, sodium percarbonate, ammonium percarbonate, sodium peroxyhydrate, calcium peroxide, sodium peroxide, sodium perborate monohydrate, sodium perborate tetrahydrate, sodium persulfate, potassium monopersulfate, perphosphate, magnesium peroxide, zinc peroxide, urea hydrogen peroxide, perhydrate of urea, thiourea dioxide, and mixtures thereof.

Another embodiment of the invention relates to the oxidizing composition as defined hereinabove, wherein the persalt is sodium percarbonate.

Another embodiment of the invention relates to the oxidizing composition as defined hereinabove, wherein said oxidizing composition is useful for preparing a tempering composition for the tempering of any kind of grains and controlling pathogens in and/or said grains. Preferably grains are selected from the group consisting of wheat, rice and corn.

Another embodiment of the invention relates to the oxidizing composition as defined hereinabove,

wherein the at least one oxidizing agent is present at a concentration varying from 0.2 to 50% by weight of the oxidizing composition, said oxidizing composition being useful for the preparation of a tempering composition for tempering of grain and controlling pathogens in and/or on the grain,
wherein the grain is wheat,
wherein the tempering composition to be prepared in addition of tempering wheat is:
1) to facilitate the milling, and/or
2) for the control of food pathogens selected from the group consisting of Shiga toxin-producing E. coli strains, and
   wherein said tempering composition preserves functionality and sensory of wheat and/or by-products resulting from the milling step, preferably by-products selected from the group consisting of bran and flour.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on the grain,
wherein the grain is wheat,
wherein the tempering composition to be prepared in addition of tempering wheat is for the control of the food pathogens selected from the group consisting of Shiga toxin-producing E. coli O157 strains, and
wherein said tempering composition preserves functionality and sensory of wheat and/or by-products resulting from the milling step, preferably by-products selected from the group consisting of bran and flour.

Another embodiment of the invention relates to the oxidizing composition as defined hereinabove,

wherein the at least one oxidizing agent is present in the oxidizing composition at a concentration varying 0.2 to 50% by weight of the oxidizing composition, said oxidizing composition being useful for the preparation of a tempering composition for tempering of grain and controlling pathogens in and/or on the grain,
wherein the grain is wheat,
wherein the tempering composition to be prepared in addition of tempering wheat is for the control of food pathogens selected from the group consisting of Shiga toxin-producing E. coli O121 strains, and
wherein said tempering composition preserves functionality and sensory of wheat and/or by-products resulting from the milling step, preferably by-products selected from the group consisting of bran and flour.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on the grain,
wherein the grain is wheat,
wherein the tempering composition to be prepared in addition of tempering wheat is for the control of the food pathogens selected from the group consisting of Salmonella strains, and
wherein said tempering composition preserves functionality and sensory of wheat and/or by-products resulting from the milling step, preferably by-products selected from the group consisting of bran and flour.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on the grain,
wherein the grain is wheat,
wherein the tempering composition to be prepared in addition of tempering wheat is for the control of the food pathogens selected from the group consisting of Listeria strains, and
wherein said tempering composition preserves functionality and sensory of wheat and/or by-products resulting from the milling step, preferably by-products selected from the group consisting of bran and flour.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on grain,
wherein the tempering composition to be prepared in addition of tempering the grain is for the control of the food pathogens selected from the group consisting of Shiga toxin-producing E. coli spp, Salmonella spp., and Listeria spp.; and
wherein said tempering composition does do not alter functionality and sensory of grain and/or by-products resulting from the milling step.

Another embodiment of the invention relates to the oxidizing composition as defined hereinabove, wherein said oxidizing composition is useful for preparing a tempering composition for tempering corn and controlling pathogens in and/or on corn.

Another embodiment of the invention relates to the oxidizing composition as defined hereinabove, wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on grain, and

wherein said tempering composition to be prepared extends the shelf life of by-products resulting from the milling step and selected from the group consisting of flour and bran but does not alter the functionality and sensory of flour and bran.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on grain,
wherein the tempering composition to be prepared in addition of tempering the grain, is for the control of the food pathogens selected from the group consisting of aerobic bacteria, yeast and mold, and
wherein said tempering composition to be prepared extends the shelf life of by-products resulting from the milling step and selected from the group consisting of flour and bran, and preserves functionality and sensory of flour and bran.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on grain,
wherein the grain is wheat,
wherein the tempering composition to be prepared in addition of tempering the wheat, is for the control of the food pathogens selected from the group consisting of aerobic bacteria, yeast and mold, and
wherein said tempering composition to be prepared prevents the wheat to be bleached or discolored, or preserves the viability of the wheat.

Another embodiment of the invention relates to the oxidizing composition as defined hereinabove,

wherein the at least one oxidizing agent is present in the oxidizing composition at a concentration varying from 0.2 to 50% by weight of the oxidizing composition, said oxidizing composition being useful for the preparation of a tempering composition for tempering of grain and controlling pathogens in and/or on grain,
wherein the grain is wheat,
wherein the tempering composition to be prepared is for the control of the food pathogens selected from the group consisting of aerobic bacteria, yeast and mold, and
wherein said tempering composition to be prepared leaves the grain free of residues.

Another embodiment of the invention relates to the oxidizing composition as defined hereinabove, wherein residues are selected from the group consisting of peracetic acid, hydrogen peroxide and acetic acid.

Another embodiment of the invention relates to the oxidizing composition as defined hereinabove, wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

Another embodiment of the invention relates to the oxidizing composition as defined hereinabove, wherein the food pathogens are selected from the group consisting of E. coli O26, E. coli O111, E. coli O103, E. coli O121, E. coli O45, and E. coli O145.

Another embodiment of the invention relates to the oxidizing composition as defined hereinabove, wherein the food pathogens are selected from the group consisting of STEC O157 and non-O157 E. coli.

Another embodiment of the invention relates to the oxidizing composition as defined hereinabove, wherein the food pathogens are selected from the group consisting of E. coli O111, E. coli O26, E. coli O45, E. coli O145, E. coli O104, E. coli O6, E. coli O51, E. coli O103, E. coli O27, and E. coli O84.

Another embodiment of the invention relates to the oxidizing composition as defined hereinabove, wherein the food pathogens is Salmonella spp.

Another embodiment of the invention relates to the oxidizing composition as defined hereinabove, wherein the food pathogens are selected from the group consisting of Salmonella typhimurium, Salmonella choleraesuis and Salmonella enteritidis.

Another embodiment of the invention relates to the oxidizing composition as defined hereinabove, wherein the food pathogens is Listeria spp.

Another embodiment of the invention relates to the oxidizing composition as defined hereinabove, wherein the food pathogens are selected from the group consisting of Listeria monocytogenes, Listeria seeligeri, Listeria ivanovii, Listeria welshimeri, Listeria marthii, Listeria innocua, Listeria grayi, Listeria fleischmannii, Listeria floridensis, Listeria aquatica, Listeria newyorkensis, Listeria comellensis, Listeria rocourtiae, Listeria weihenstephanensis, Listeria grandensis, Listeria riparia and Listeria booriae.

Another embodiment of the invention relates to the oxidizing composition as defined hereinabove, wherein the food pathogens are selected from the group consisting of Salmonella spp, Listeria spp. and E. coli spp.

Another embodiment of the invention relates to the oxidizing composition as defined hereinabove, wherein the food pathogens are selected from the group consisting of Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

Another embodiment of the invention relates to the oxidizing composition as defined hereinabove, said oxidizing composition being useful for the preparation of a tempering composition for tempering of grain and controlling pathogens in and/or on grain, wherein during the subsequent milling step the tempering composition further cleans and sanitizes mill systems to thus prevent cross-contamination and reduce the load of microorganisms in the milling systems.

Another embodiment of the invention relates to the method for sanitizing mill systems, said method comprising a step of contacting mill systems with an oxidizing composition as defined hereinabove. Preferably, in such a case, mill systems are first manually cleaned to remove dust, residues, biofilms, bacteria, yeast and mold, and then the oxidizing composition defined hereinabove is sprayed or fogged on mill systems to reduce/control the pathogens count. Preferably, the pathogens may comprise at least one of bacteria, yeasts and/or molds), and mill systems may comprise at least one of bins, pipings, milling equipment, etc.

### DETAILED DESCRIPTION OF PREFERRED EMBODIMENTS OF THE INVENTION

There will be provided hereinafter experimental tests illustrating particularly preferred embodiments of the invention

Protocols, results and conclusions

### Example 1

### Introduction

An organic flour mill was selected in Quebec Canada to conduct a trial in order to determine the efficacy of the organic novel composition identified as Neo-Temper which is composed of a stabilized peracetic acid generated by the reaction of acetic acid and hydrogen peroxide. More particularly this Neo-Temper tailored for the tempering-holding-milling phases in flour mills was compared to citric and lactic acids as antibacterial agents. Neo-Temper has the following formulation:

| | | | Percentage | |
|---|---|---|---|---|
| Ingredients | | CAS # | (w/w) | |
| Distilled Water | | 7732-18-5 | 34.5% | |
| Acetic Acid 92% | Post-reaction: | 64-19-7 | 13.5% | 5% |
| Hydrogen | Generates | 7722-84-1 | 50.0% | peracetic |
| Peroxide 50% | guaranteed | | | acid |
| | minimum | | | |
| | 50,000 ppm | | | |
| | active | | | |
| | pre-formed | | | |
| | peracetic | | | |

| | | |
|---|---|---|
| | acid (CAS | |
| | #79-21-0) | |
| Sulphuric Acid 96% | 7664-93-9 | 1.1% |
| Dequest 2010: | 2809-21-4 | 0.9% |
| 1-hydroxyethylidene- | | |
| 1,1-diphosphonic acid | | |
| TOTAL: | | 100% w/w |

Guarantee: Minimum 5% active

### Protocol

Tempering of wheat is obtained by adding water to the wheat at a rate to reach the desired moisture content of wheat. Concerning the following experiments, a determined volume of Neo-Temper was substituted to a corresponding volume of the tempering-water (e.g. if 20 liters of tempering are normally required for the tempering of wheat, then the tempering is achieved with a mix of 18 liters of water and 2 liters an oxidizing composition (i.e. 2 liters of Neo-Temper). The holding time for the following experiments was 16 hours before milling, and the usual amount of tempering-water (before any partial substitution with an oxidizing composition) is about 40 liters/tonne of wheat.
Treatment 1—Neo-Temper 2 L: 2 Liters of Neo-Temper (substituting 2 L water) were added per ton of wheat during water-addition in the tempering step.
Treatment 2—Neo-Temper 4 L: 4 Liters of Neo-Temper (substituting 4 L water) were added per ton of wheat during water-addition in the tempering step.
Treatment 3—Neo-Temper+Surfactant 4 L: 4 Liters of Neo-Temper containing an organic surfactant (substituting 4 L water) were added per ton of wheat during water-addition in the tempering step. The surfactant is Tween 20 (a polysorbate surfactant).
Treatment 4—Citric acid: 2 Kg of Citric acid were added per ton of wheat during water-addition in the tampering step.
Treatment 5—Lactic acid: 2 Liters of lactic acid (substituting 2 L water) were added per ton of wheat during water-addition in the tempering step.
Treatment 6—water: Only water was added as usual with no organic antimicrobial solution.

The results are described in Table 1.

TABLE 1

| | | TRT 1 | TRT 2 | TRT 3 | | | |
|---|---|---|---|---|---|---|---|
| | | Neo- | Neo- | Neo-Temper | TRT 4 | TRT 5 | TRT6 |
| | | Temper | Temper | 4L + | Citric | Lactic | Control |
| | | 2L | 4L | surf. | acid | acid | H₂O |
| FOSS | % Proteins | 11.38 | 11.46 | 11.34 | 11.32 | 11.54 | 11.37 |
| | % humidity | 13.1 | 13.2 | 13.4 | 13.5 | 12.8 | 12.9 |
| | % Ash | 0.49 | 0.52 | 0.49 | 0.48 | 0.51 | 0.48 |
| | % gluten | 31.62 | 31.17 | 31.56 | 31.97 | 31.83 | 31.91 |
| | % absorption | 59.98 | 60.49 | 59.8 | 59.82 | 60.85 | 60.68 |
| IDC | second | 346 | 348 | 366 | 367 | 338 | 356 |
| MIXOLAB | % H₂O | 59.9 | 58.8 | 60.1 | 60.1 | 59.6 | 60.2 |
| | Stability (min) | 14 | 21.5 | 15 | 13.5 | 17.5 | 13.5 |
| | Aff 12 min (Nm) | 0.005 | 0.016 | 0.018 | 0.022 | 0.022 | 0.01 |
| | PMT (second) | 214 | 178 | 202 | 185 | 186 | 176 |
| GPT | BEM (Brabender | 54 | 51 | 51 | 51 | 52 | 54 |
| | unit) | | | | | | |
| | AM/BM (second) | 48 | 47 | 47 | 46 | 45 | 49 |
| | PM/AM (second) | 48 | 42 | 46 | 45 | 42 | 48 |
| | % Gluten—Wet | 35.4 | 34.6 | 34.6 | 34.8 | 34.6 | 35.7 |
| | % Protein content | 11.6 | 11.2 | 11.2 | 11.3 | 11.2 | 11.7 |
| | % Hydration | 60 | 59 | 60 | 60 | 59.5 | 60 |
| VMI | Time (second) | 54 | 62 | 54 | 54 | 43 | 55 |
| | Temperature (° C.) | 24.8 | 24 | 22.5 | 23.6 | 22.6 | 23.8 |
| | Strength (Watt per | 517 | 510 | 493 | 510 | 509 | 520 |
| | hour) | | | | | | |
| | Energy (Watt per | 15.6 | 16.8 | 7.8 | 15.8 | 14.9 | 8.8 |
| | hour) | | | | | | |
| EXTENSO 1 | Energy (cm-2) | 134 | 126 | 131 | 130 | 148 | 139 |
| | Resistance (EU) | | | | | | |
| | (EU = extensograph | 458 | 440 | 429 | 459 | 421 | 472 |
| | petrin outle tunit) | | | | | | |
| | Extensibility (mm) | 154 | 155 | 158 | 149 | 169 | 156 |
| | Maximum (mm) | 700 | 652 | 658 | 705 | 712 | 720 |
| | Ratio (EU/mm) | 3 | 2.8 | 2.7 | 3.1 | 2.5 | 3 |
| | Ratio Max (EU/mm) | 4.5 | 4.2 | 4.2 | 4.7 | 4.2 | 4.6 |
| EXTENSO 2 | Energy (cm-2) | 141 | 125 | 141 | 136 | 124 | 128 |
| Division | Resistance (EU) | 744 | 686 | 667 | 613 | 532 | 636 |
| after fermen | | | | | | | |
| | Extensibility (mm) | 125 | 120 | 135 | 136 | 136 | 127 |
| | Maximum (mm) | 926 | 880 | 858 | 809 | 734 | 861 |
| | Ratio (EU/mm) | 6 | 5.7 | 4.9 | 4.5 | 3.9 | 5 |
| | Ratio Max (EU/mm) | 7.4 | 7.3 | 6.4 | 6 | 5.4 | 6.8 |
| EXTENSO 1 | Energy (cm-2) | 80 | 82 | 103 | 75 | 64 | 74 |
| 3 h 30 after | Resistance (EU) | 240 | 257 | 222 | 207 | 200 | 240 |
| fermen. | | | | | | | |
| | Extensibility (mm) | 180 | 185 | 217 | 193 | 179 | 174 |
| | Maximum (mm) | 329 | 319 | 360 | 298 | 270 | 303 |
| | Ratio (EU/mm) | 1.3 | 1.4 | 1 | 1.1 | 1.1 | 1.4 |
| | Ratio Max (EU/mm) | 1.8 | 1.7 | 1.7 | 1.5 | 1.5 | 1.7 |
| size poolish | cm | 13.8 | 13.8 | 14.1 | 14.2 | 15.1 | 12.2 |
| Bread size- | mm | 157 | 165 | 132 | 158 | 146 | 149 |
| mold | | | | | | | |

Conclusion: Neo-Temper (2 L per Ton) had no impact on flour functionality. Neo-Temper (4 L per Ton) was accepted by the organic mill but borderline. Neo-Temper+surfactant (i.e. Tween 20, a polysorbate surfactant) was rejected for its effect on energy, resistance and extensibility of the bread. Likewise, citric acid and Lactic acid were both rejected for their negative impact on resistance. Moreover, Neo-Temper 2 L per ton and 4 L per ton had no negative impact on sensory and physical characteristics.

For those skilled in the art, it was unusual that both citric and lactic acid (2 kg and 2 L per ton respectively) had a negative impact on functionality whereas Neo-Temper 2 L and even up to 4 L per ton of wheat) had no negative impact on functionality noting that Neo-Temper contains not only an acid component like citric and lactic acid but it contains a strong acidic oxidizer (hydrogen peroxide) at high percentage.

Also, for those skilled in the art, it was unusual that Neo-Temper 2 L and even up to 4 L per ton of wheat) had no negative impact on sensory noting that Neo-Temper has a strong acetic acid (Vinegar) smell.

Moreover, there is no drying step before milling which usually helps in odor removal. The presence of peracetic acid and hydrogen peroxide did not bleach or discolor the wheat and had no negative effect on the physical characteristics and taste.

Finally, for those skilled in the art, it was unusual that the surfactant (i.e. Tween 20, a polysorbate surfactant) had a such negative impact on gluten and extensibility.

Also, it was surprising for those skilled in the art that oxidizing agents such as peracetic acid and hydrogen peroxide did not ruin the food (sensory, functionality) and that the hold time during the tempering phase helps in the reduction of microorganisms but did not damage the food. It was also surprising that hydrogen peroxide performed better than other oxidizing agents and acids.

Also, it was surprising for those skilled in the art that this composition provides control of pathogens while maintaining functionality. That was not obvious to a person of ordinary skill in the art. It was surprising that the flour and bran do not smell like vinegar. The fact that it is rate-sensitive and that 4 L of the composition hurts functionality, while 2-3 L does not is also surprising.

### Example 2

### Introduction

Since Neo-Temper 2 L per ton had no negative impact on functionality, physical characteristics and sensory whereas Neo-Temper 4 L per ton was borderline, the objective of this experiment was to repeat the treatments and include Neo-Temper 3 L per ton.

### Protocol

Concerning the following experiments, a determined volume of Neo-Temper was substituted to a corresponding volume of the tempering-water (e.g. if 20 liters of tempering are normally required for the tempering of wheat, then the tempering is achieved with a mix of 17 liters of water and 3 liters an oxidizing composition (i.e. 3 liters of Neo-Temper). The for the following experiments was 16 hours before milling, and the usual amount of tempering-water (before any partial substitution with an oxidizing composition) was about 20 liters/tonne of wheat. More particularly, a flow of the Neo-Temper was directly injected with a metering pump (ProMinent® into the flow of tempering-water going to tempering bins. A total solution of 20 L including the tempering-water and the oxidizing composition (New-Temper) were sprayed on 1 ton of wheat in the tempering bins.
Treatment 1—Neo-Temper 2 L: 2 Liters of Neo-Temper (substituting 2 L water) were added per ton of wheat during water-addition in the tampering step.
Treatment 2—Neo-Temper 3 L: 3 Liters of Neo-Temper (substituting 3 L water) were added per ton of wheat during water-addition in the tempering step.
Treatment 3—water: Only water was added as usual with no organic antimicrobial solution.

The results are described in Table 2.

TABLE 2

| TRT 1- | TRT 2- | TRT 3- | | |
|---|---|---|---|---|
| 20.06.2017 | 20.06.2017 | 20.06.2017 | | |
| Control | 2LNeo- | 3L Neo- | | |
| | Temper | Temper | | |
| 11.25 | 11.28 | 11.11 | % proteins | FOSS |
| 14.92 | 14.26 | 14.6 | % humidity | |
| 0.47 | 0.52 | 0.43 | % Ash | |
| 29.79 | 30.7 | 31.04 | % gluten | |
| 59.41 | 59.33 | 58.95 | % absorption | |
| 342 | 362 | 355 | second | IDC |
| 58.6 | | | % H₂O | MIXOLAB |
| 19.5 | | | Stability (min) | |
| 0.065 | | | Weakening 12 | |
| | | | min (Nm). | |
| 58.6 | 58.6 | 58.6 | % H₂O | MIXOLAB+ |
| −0.086 | −0.1 | −0.102 | Alpha (Nm/min) | |
| 0.432 | 0.374 | 0.368 | Beta (Nm/min) | |
| 0 | −0.012 | −0.03 | Y (Nm/min) | |
| 169 | 174 | 162 | PMT (second) | GPT |
| 51 | 52 | 52 | BEM (Brabender | |
| | | | unit) | |
| 35 | 47 | 37 | AM/BM (second) | |
| 46 | 44 | 45 | PM/AM (second) | |
| 34.9 | 35 | 34.7 | % Gluten Humid | |
| 11.3 | 11.4 | 11.3 | % proteins | |

Conclusion: Neo-Tem per (2 L and 3 L per Ton) had no impact on functionality. The effect of Neo-Temper (2 L and 3 L per ton) is almost similar to the untreated as shown in Table 2. Moreover, Neo-Temper (2 L and 3 L per ton) had no negative impact on sensory and physical characteristics.

### Example 3

### Introduction

Since Neo-Temper (2 L and 3 L per ton) had no impact on functionality, sensory and physical characteristics, it was worth investigating the efficacy of Neo-Temper in reducing E. coli strains on wheat.

### Protocol:

### Preparation of Inoculant:

3 strains of E. coli O157:H7 were each grown to stationary phase in 50 mL BHI broth (35C with shaking at 150 rpm, 18-24 h). Cells were pelleted by centrifugation at 4,000×g for 10 minutes and washed 3× with 50 mL of 0.1% peptone water. Cells were resuspended in 8 mL of 0.1% peptone water and pooled to obtain 2 inoculum cocktails for O157:H7 strains and for O121 strains. The estimated cell concentration was 3.6E+08 cfu/mL for O157:H7.

Sample Inoculation

2 mL of the inoculum was mixed with 100 g of Wheat with vigorous mixing and allowed to dry/acclimatize (exposed to air) for 24 hours in a biohood cabinet at 22C, Followed by treatment or no treatment of the spiked samples with the various solutions. Samples were analysed 24 hours after treatment.

### Enumeration

Samples were mixed with 1:2 ratio of sample to mTSB enrichment broth. Additional 10-fold serial dilutions from the 1/2 dilution were prepared in 0.1% peptone water. For enumeration, 1 mL (0.33 ml×3 plates) from the 1/2 dilution and 0.1 mL of subsequent 10-fold dilutions were spread plated onto Rainbow Agar. Enumeration results are reported in cfu/g (the minimum detection limit was 2 cfu/g). For presence/absence the initial 1/2 diluted sample in mTSB was enriched at 42° C. for 18-24 hours, 1 mL was then subjected to immunomagnetic separation using O157 coated magnetic beads and plated onto ChromAgarO157 and CR-SMAC for O157:H7 inoculated samples. Suspect colonies were confirmed via the use of antisera or PCR.

Samples used in this trial were initially negative for the presence of STEC as determined by PCR and thus were considered to be free of the target organism prior to inoculation. Results are reported in Table 3.

TABLE 3
Efficacy of Neo-Temper on wheat infected with STEC

| Treatments | Grain | CFU/ml |
|---|---|---|
| WHEAT O157 UT-1 | Wheat | 680000 |
| WHEAT O157 UT-2 | Wheat | 360000 |
| WHEAT O157 UT-3 | Wheat | 152000 |
| WHEAT O157 4 L/ton A-1 | Wheat | <2 |
| WHEAT O157 4 L/ton A-2 | Wheat | <2 |
| WHEAT O157 4 L/ton A-3 | Wheat | <2 |
| WHEAT O157 4 L + surf/ton | Wheat | <2 |
| B-1 | | |
| WHEAT O157 4 L + surf/ton | Wheat | <2 |
| B-2 | | |
| WHEAT O157 4 L + surf/ton | Wheat | <2 |
| B-3 | | |
| WHEAT O157 2 L/ton C-1 | Wheat | 2 |
| WHEAT O157 2 L/ton C-2 | Wheat | 4 |
| WHEAT O157 2 L/ton C-3 | Wheat | <2 |

### Conclusion:

Growth potential of E. coli of serotype O157 on artificially inoculated Wheat and treated with three Neo-Temper formulations at a rate: 4 mL/100 g (A), 4 mL+surf/100 g (B) and 2 mL/100 g (C) sample with a 24 hour hold period compared to untreated controls. This experiment was done in triplicate. The Untreated O157:H7 control was 5.52 log cfu/g. Below are the results of treatments A, B and C.

A) Growth potential of E. coli O157:H7 on Wheat treated with Neo-Temper (4 L) mixed in 36 L water per ton wheat was −5.22 to −5.52 log cfu/g
B) Growth potential of E. coli O157:H7 on Wheat treated with Neo-Temper (4 L) containing 0.2 L surfactant (i.e. Tween 20, a polysorbate surfactant) mixed in 36 L water was −5.22 to −5.52 log cfu/g
C) Growth potential of E. coli O157:H7 on Wheat treated with Neo-Temper (2 L) mixed in 38 L water was −5.07 log cfu/g

### Example 4

In the following example, the following oxidizing compositions were used: Neo-Temper and Neo-Temper with hydrogen peroxide. Those oxidizing composition have the following formulations:

Neo-Temper has the following formulation:

| Ingredients | | CAS # | Percentage (w/w) | |
|---|---|---|---|---|
| Distilled Water | | 7732-18-5 | 34.5% | |
| Acetic Acid 92% | Post-reaction: | 64-19-7 | 13.5% | 5% |
| Hydrogen | Generates | 7722-84-1 | 50.0% | peracetic |
| Peroxide | guaranteed | | | acid |
| | minimum | | | |
| | 50,000 ppm | | | |
| | active | | | |

pre-formed
peracetic
acid (CAS
#79-21-0)

| | | |
|---|---|---|
| Sulphuric Acid 96% | 7664-93-9 | 1.1% |
| Dequest 2010: | 2809-21-4 | 0.9% |
| 1-hydroxyethylidene- | | |
| 1,1-diphosphonic acid | | |

| | |
|---|---|
| TOTAL: | 100% w/w |

Guarantee: Minimum 5% active
Neo-Temper+hydrogen peroxide

The above-mentioned Neo-Temper formulation and 35% hydrogen peroxide were simultaneously injected with a metering pump (ProMinent®) into a flow of tempering-water going to tempering bins.

Efficacy of Neo-Temper Against Shiga-Toxin Producing E. coli (STEC) and its Non-Pathogenic Surrogate on Wheat

The efficacy of “Neo-Temper” and “Neo-Temper with hydrogen peroxide” was tested in the following experiments:
1. Pathogen challenge test—Efficacy of Neo-Temper (with 16 h hold time post-treatment) in eliminating a cocktail of STEC Escherichia coli on wheat
2. Surrogate compatibility test—Efficacy of Neo-Temper (with 8 h hold time post treatment) in eliminating a Shiga-toxin producing Escherichia coli cocktail compared to a non-pathogenic E. coli cocktail on irradiated wheat
3. Test on STEC surrogate—Efficacy of Neo-Temper with hydrogen peroxide (with 16 h hold post-treatment) in eliminating a non-pathogenic E. coli cocktail (STEC surrogate) on wheat

### Procedure:

1. Pathogen challenge test:

Wheat samples (200 g each) were inoculated with a seven strain cocktail of shiga-toxin producing E. coli (STEC) (serotypes O26:H11, O103:H2, O11:NM, O121:H19, O145:NM, 045:H2 and O157:H7) each grown in BHI broth (35° C. with shaking at 150 rpm, for 18-24 h). Cultures were pooled and cells were pelleted by centrifugation at 4,000 g for 10 minutes and washed 3 times with 0.1% peptone water. Cells were resuspended in 0.1% peptone water. Two mL of the cocktail inoculum was vigorously mixed with 100 g of wheat (N=5) and allowed to dry/acclimatize (exposed to air) for 24 hours in a biohood cabinet at 22° C., followed by treatment or no treatment of inoculated samples with various solutions. Treated samples were held in biosafety cabinet at 22° C. for 16 h.

The solutions tested were:
Solution A: 2 L Neo-Temper in 21 L H₂O (applied at 23 L/tonne):
Solution B: 2 L Neo-Temper in 68 L H₂O (applied at 70 L/tonne)
Solution C: 3 L Neo-Temper in 20 L H₂O (applied at 23 L/tonne)
Solution D: 3 L Neo-Temper in 67 L H₂O (applied at 70 L/tonne)

More particularly, the Neo-Temper was injected with a metering pump (ProMinent®) into tempering-water, and then sprayed via nozzles on wheat and held in tempering bins for at least 4 hours.

The E. coli counts in CFU/g of treated or untreated samples were obtained as follows: samples were first homogenized/mixed with 1:2 ratio of sample to 0.1% peptone water and stomached for 2 min. Additional 10-fold serial dilutions from the 1/2 dilution were also prepared in 0.1% peptone water. For enumeration, 1 mL (0.33 ml×3 plates) from the 1/2 dilution and subsequent 10-fold dilutions were spread plated onto TSA. Enumeration results are reported in CFU/g (the minimum detection limit was 2 CFU/g). This experiment was repeated independently three times for a total of three biological replicates with 5 replicates in each biological replicate (total N=15). Enumeration results are reported in log CFU/g (minimum detection limit of 2 CFU/g). Samples used were initially negative for the presence of STEC as determined by PCR and thus were considered free of the target organism prior to inoculation.

2. Surrogate compatibility test:

Irradiated wheat samples (200 g each) were inoculated with either 1) a seven strain cocktail of shiga-toxin producing E. coli (STEC) (serotypes O26:H11, O103:H2, O11:NM, O121:H19, O145:NM, 045:H2 and O157:H7) or 2) a non-pathogenic E. coli cocktail composed of ATTC strains (BAA-1427, BAA-1428, BAA-1429, BAA-1430 and BBA-1431) each grown in BHI broth (35° C. with shaking at 175 rpm, for 18-24 h). Cultures were pooled and cells were pelleted by centrifugation at 4,000 g for 10 minutes and washed 3 times with 0.1% peptone water. Cells were resuspended in 0.1% peptone water. Two mL of the cocktail inoculum was vigorously mixed with 100 g of wheat (N=3) and allowed to dry/acclimatize (exposed to air) for 24 hours in a biohood cabinet at 22° C., followed by treatment or no treatment of inoculated samples with 7 mL/100 g (70 L/tonne) of the solution D (3 L of Neo-Temper:67 L of water). Treated samples were held in biosafety cabinet at 22° C. for 8 h. E. coli counts in CFU/g of treated or untreated samples were obtained as follows: Samples were first homogenized/mixed with 1:2 ratio of sample to 0.1% peptone water and stomached for 2 min. Additional 10-fold serial dilutions from the 1/2 dilution were also prepared in 0.1% peptone water. For enumeration, 1 mL (0.33 ml×3 plates) from the 1/2 dilution and subsequent 10-fold dilutions were spread plated onto TSA. Enumeration results are reported in CFU/g (the minimum detection limit was 2 CFU/g). This experiment was repeated independently three times (N=3). Enumeration results are reported in log CFU/g (minimum detection limit of 2 CFU/g). The wheat utilized in these trial was confirmed to be free of background microbiota.

3. Test on STEC surrogate:

Wheat samples of 2 kg were inoculated with a non-pathogenic E. coli cocktail composed of ATTC strains (BAA-1427, BAA-1428, BAA-1429, BAA-1430 and BBA-1431). The strains were grown separately for 18 hours at 35° C. and mixed the day of the inoculation. The inoculum was applied while mixing the grain vigorously in a kitchen mixer at a rate of 30 mL per kg of wheat, and allowed to dry/acclimatize (exposed to air) for 24 hours in a biohood cabinet at 22° C. Then, 3 samples of 45 g each, were withdrawn for enumeration. These constituted an analysis of untreated controls (UTC) to determine the concentration of surviving bacteria before treatment. The remaining wheat was treated at a rate of 70 L/tonne of a tempering composition (3 L of Neo-Temper:10 L of 35% hydrogen peroxide:57 L of water). After 16 hour of the treatment, 1 kg of the treated wheat was milled into flour in a laboratory scale miller, and 3 samples of flour, 45 g each, were taken for E. coli enumeration. In addition, the flour samples were stored and E. coli enumeration was performed at 1, 2 and 4 weeks after the treatment. This experiment was repeated independently three times for a total of three biological replicates with 5 replicates in each biological replicate (total N=15). E. coli counts in CFU/g of treated or untreated samples were obtained as follows: Samples were first homogenized/mixed with 1:2 ratio of sample to 0.1% peptone water and stomached for 2 min. Additional 10-fold serial dilutions from the 1/2 dilution were also prepared in 0.1% peptone water. For enumeration, 1 mL (0.33 ml×3 plates) from the 1/2 dilution and subsequent 10-fold dilutions were spread plated onto Brilliance E.coli coliform selective agar. Enumeration results are reported in CFU/g (the minimum detection limit was 2 CFU/g).

Effect of Neo-Temper (with 16 hour Hold Post-treatment)
on STEC Cocktail Inoculated on Wheat
Effect of Neo-Temper (with 16 hour Hold Post-treatment)
on STEC Cocktail Inoculated on Wheat

| | Recovered STEC population | | | Average | Log |
|---|---|---|---|---|---|
| | (Avg.,N = 5 Each trial) | | | | |
| | (log CFU/g ± SD) | | | (log CFU/g ± | Reduction |
| Sample | Trial #1 | Trial #2 | Trial #3 | SD) | (log CFU/g) |
| UTC | 6.54 ± | 5.53 ± | 5.63 ± | 5.90 ± | N/A |
| | 0.29 | 0.11 | 0.23 | 0.55 | |
| Solution | 3.17 ± | 2.57 ± | 3.07 ± | 2.94 ± | 2.96 |
| A 23 | | | | | |
| L/tonne | | | | | |
| (2 L Neo- | | | | | |
| Temper | 0.28 | 0.43 | 0.06 | 0.32 | |
| in 21 | | | | | |
| L H₂O) | | | | | |
| Solution | 3.75 ± | 2.54 ± | 2.51 ± | 2.93 ± | 2.97 |
| B 70 | | | | | |
| L/tonne | 0.13 | 0.14 | 0.18 | 0.71 | |
| (2 L Neo- | | | | | |
| Temper | | | | | |
| in 68 | | | | | |
| L H₂O) | | | | | |
| Solution | 3.20 ± | 3.50 ± | 3.18 ± | 3.29 ± | 2.61 |
| C 23 | | | | | |
| L/tonne | 0.13 | 0.08 | 0.16 | 0.18 | |
| (3 L Neo- | | | | | |
| Temper | | | | | |
| in 20 | | | | | |
| L H₂O) | | | | | |
| Solution | 3.04 ± | 2.06 ± | 2.46 ± | 2.52 ± | 3.38 |
| D 70 | | | | | |
| L/tonne | 0.17 | 0.28 | 0.27 | 0.49 | |
| (3 L Neo- | | | | | |
| Temper | | | | | |
| in 67 | | | | | |
| L H₂O) | | | | | |

SD = standard deviation; UTC = untreated control (inoculated)

### Results

### 1. Pathogen Challenge Test:

The table below shows the efficacy of different solutions against a seven-strain STEC cocktail. The reported initial level of STEC (5.90±0.55 Log CFU/g) belongs to the untreated wheat samples that were enumerated 24 hour after inoculation. The results show that population of STEC dropped to 2.94±0.32, 2.93±0.71, 3.29±0.18 and 2.52±0.49 log CFU/g after treatment with solutions A, B, C, or D, respectively, producing 2.96, 2.97, 2.61, and 3.38 log reduction on STEC. Solution D (3 L Neo-Temper in 67 L H₂O), applied at 70 mL/kg (70 L/tonne), was the most effective against STEC. Therefore it was chosen for future testing.

### 2. Surrogate Compatibility Test:

The table below shows the comparison of the efficacy of a tempering composition (3 L of Neo-Temper:67 L of water), on a seven-strain STEC cocktail and a five strain cocktail of non-pathogenic E. coli on wheat. The initial counts of the non-pathogenic E. coli cocktail were about 0.7 log CFU/g lower than the STEC cocktail. However, the average log CFU/g reduction values (calculated as the difference between the average UTC log CFU/g and the treated sample's log CFU/g), were comparable after 8 h treatment with Neo-Temper, with no statistically significant difference (P>0.05) determined by one-way ANOVA (alpha=0.05).

Effect of tempering composition ((3 L of Neo-Temper: 67 L of water) at
70 L/tonne with 8 hour Hold Post-treatment) on an STEC Cocktail and a
Non-Pathogenic E. coli cocktail on Wheat
Effect of tempering composition ((3 L of Neo-Temper: 67 L of water) at
70 L/tonne with 8 hour Hold Post-treatment) on an STEC
Cocktail and a Non-Pathogenic E. coli cocktail on Wheat

| | Average | Average Log |
|---|---|---|
| | Recovered ± SD | Reduction |
| Sample | (log CFU/g) | (log CFU/g) |
| Wheat inoculated with | 6.29 ± 0.19 | — |
| STEC cocktail, UTC | | |
| Wheat inoculated with | 4.20 ± 0.31 | 2.09 |
| STEC cocktail, Treated | | |
| Wheat inoculated with non- | 5.60 ± 0.31 | — |
| pathogenic E. coli cocktail, UTC | | |
| Wheat inoculated with non- | 3.64 ± 0.31 | 1.96 |
| pathogenic E. coli cocktail, Treated | | |

SD = standard deviation; UTC = untreated control (inoculated)

3. Test on STEC surrogate:

The table below shows the efficacy of a tempering composition (3 L of Neo-Temper:10 L of 35% hydrogen peroxide:57 L of water) against a the non-pathogenic E. coli cocktail, which previously demonstrated to be a suitable surrogate for STEC on wheat. The treatment was able to achieve 3.36 log CFU/g reduction on the E. coli cocktail on the flour obtained from the treated wheat, after 16 h hold. In addition, the counts of E. coli in the flour decreased consistently after 1, 2 and 4 weeks of the treatment, achieving 4.11, 4.70 and 5.31 log CFU/g reductions, respectively

Effect of the tempering composition ((3 L of Neo-Temper:10 L of 35%
hydrogen peroxide:57 L of water) at 70 L/tonne, with 16 hour Hold
Post-treatment) on a Non-Pathogenic E. coli cocktail on Wheat
Effect of the tempering composition ((3 L of Neo-Temper: 10 L of 35%
hydrogen peroxide: 57 L of water) at 70 L/tonne, with 16 hour Hold
Post-treatment) on a Non-Pathogenic E. coli cocktail on Wheat

| | WHEAT | | |
|---|---|---|---|
| | Average | | Average |
| | Untreated | | Log |
| | Control | FLOUR | Reduction |
| | SD (log | Average Recovered | (log |
| Sample | CFU/g) | SD (log CFU/g) | CFU/g) |
| Week 0 (16 h hold) | 5.82 ± 0.21 | 2.46 ± 0.11 | 3.36 |
| Week 1 | NA | 1.70 ± 0.37 | 4.11 |
| Week 2 | | 0.55 ± 0.24 | 4.70 |
| Week 4 | | 0.84 ± 0.53 | 5.31 |

SD = standard deviation

### CONCLUSIONS

The pathogen challenge test demonstrated that Solution D (3 L of Neo-Temper:67 L of water) applied at 70 L/tonne, was the most effective against the STEC cocktail, achieving 3.38 log CFU/g reduction with 16 h hold time post-treatment. Therefore, this formula and rate were used for the rest of the tests.

The surrogate compatibility test proved that the non-pathogenic cocktail of E. coli is a suitable surrogate for STEC for testing the efficacy the tempering composition (3 L of Neo-Temper:67 L of water) on wheat, as indicated by the consistency in the log CFU/g reductions achieved 8 h after the treatment at 70 L/tonne.

The efficacy of the tempering composition (3 L of Neo-Temper:10 L of 35% hydrogen peroxide:57 L of water) applied at 70 L/tonne on the STEC surrogate cocktail on wheat was demonstrated by achieving >3 log CFU/g reduction 16 h after the treatment and by the increase in the log CFU/g reduction to >5, after 4 weeks of the treatment.

It is understood that the examples and embodiments described herein are for illustrative purposes only and that various modifications or changes in light thereof will be suggested to persons skilled in the art and are to be included within the present invention and scope of the appended claims.

1-139. (canceled)

140. A tempering composition for tempering grain in a tempering step and controlling pathogens susceptible to be present in and/or on said grain during the tempering step of the grain, said tempering step being eventually carried out before subjecting the tempered grain to a milling step;
wherein said tempering composition comprises tempering-water and an oxidizing composition comprising at least one oxidizing agent and/or a precursor thereof, and eventually at least one an agriculturally acceptable excipient and/or at least one additive;
wherein the at least one oxidizing agent represents from 0.01 to 50% by weight of the oxidizing composition;
herein the at least one oxidizing agent comprises:
a) liquid peracid and/or in-situ generated peracid; and/or
b) liquid hydrogen peroxide and/or hydrogen peroxide released from a hydrogen peroxide precursor; and
wherein the tempering composition is to be applied to the grain at a rate varying from 20 to 80 liters of the tempering composition per ton of grain.

141. The tempering composition according to claim 140, wherein the oxidizing composition is admixed with the tempering-water in a weight ratio oxidizing composition:tempering-water varying from 4:16 to 2:78.

142. The tempering composition according to claim 140, wherein the tempering composition is suitable for contacting the grain for a period varying from 2 to 48 hours.

143. The tempering composition according to claim 140, wherein the at least one oxidizing agent comprises:
a) a liquid peracetic acid and/or in-situ generated peracetic acid;
b) optionally a liquid hydrogen peroxide and/or a hydrogen peroxide released from a hydrogen peroxide precursor;
c) water; and
c) optionally at least one additive and/or at least one agriculturally acceptable excipient.

144. The tempering composition according to claim 140, wherein the liquid peracetic acid is obtained from the reaction of acetic acid with hydrogen peroxide.

145. The tempering composition according to claim 1, wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

146. The tempering composition according to claim 140, wherein the food pathogens are selected from the group consisting of Salmonella spp, Listeria spp., E. coli spp., Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

147. A method for tempering grain and for the control of pathogens susceptible to be present in and/or on said grain during a tempering step of the grain with a tempering composition to thereby obtain a tempered grain, said tempering step being eventually carried out before subjecting the tempered grain to a milling step;
wherein said method comprises a step of contacting the tempering composition with the grain to thereby obtain the tempered grain,
wherein the tempering composition comprises tempering-water and an oxidizing composition comprising at least one oxidizing agent and/or a precursor thereof, and eventually at least one an agriculturally acceptable excipient and/or at least one additive;
wherein the at least one oxidizing agent represents 0.01 to 50% by weight of the oxidizing composition;
wherein the at least one oxidizing agent comprises:
a) liquid peracid and/or in-situ generated peracid; and/or
b) liquid hydrogen peroxide and/or hydrogen peroxide released from a hydrogen peroxide precursor; and
wherein the tempering composition is applied to the grain at a rate varying from 20 to 80 liters of the tempering composition per ton of grain.

148. The method according to claim 147, wherein the oxidizing composition is admixed with the tempering-water in a weight ratio oxidizing composition:tempering-water varying from 4:16 to 2:78.

149. The method according to claim 147, wherein the tempering-water, the oxidizing composition comprising the at least one oxidizing agent and/or a precursor thereof, and eventually the at least one an agriculturally acceptable excipient and/or further at least one additive, are mixed together in a mixing apparatus.

150. The method according to claim 147, wherein the tempering composition is contacted with grain in the tempering step by pumping, fumigating, spraying, misting or vaporizing said tempering composition on the grain.

151. The method according to claim 150, wherein a flow of the tempering composition is sprayed on a flow of the grain, and then the mixture of the grain and the tempering composition is held in a tempering tank for a period varying from 2 to 48 hours.

152. The method according to claim 147, wherein the at least one oxidizing agent comprises:
a) a liquid peracetic acid and/or an in-situ generated peracetic acid;
b) optionally a liquid hydrogen peroxide and/or a hydrogen peroxide released from a hydrogen peroxide precursor;
c) water; and
d) optionally at least one additive and/or at least one agriculturally acceptable excipient.

153. The method according to claim 147, wherein the liquid peracetic acid obtained from the reaction of acetic acid with hydrogen peroxide.

154. The method according to claim 147, wherein the food pathogens are selected from the group consisting of E. coli cocktail, Shiga toxin-producing E. coli (STEC), Verocytotoxin (VTEC), enterohemorrhagic E. coli (EHEC), Enterotoxigenic E. Coli (ETEC), Enteropathogenic E. coli (EPEC), Enteroaggregative E. coli (EAEC), Enteroinvasive E. coli (EIEC) and Diffusely adherent E. coli (DAEC).

155. The method according to claim 147, wherein the food pathogens are selected from the group consisting of Salmonella spp, Listeria spp., E. coli spp., Bacillus cereus, Campylobacter jejuni, Clostridium botulinum, Clostridium perfringens, Cryptosporidium, Cyclospora cayetanensis, Hepatitis A, Noroviruses, Shigella spp., Staphylococcus aureus, Vibrio parahaemolyticus and Vibrio vulnificus.

156. The method according to claim 147, wherein during the subsequent milling step the tempering composition further cleans and sanitizes mill systems to prevent cross-contamination and reduce the load of microorganisms in said mill systems.

157. An oxidizing composition useful for the preparation of a tempering composition as defined in claim 140, wherein the oxidizing composition comprises at least one oxidizing agent and/or a precursor thereof, and eventually at least one an agriculturally acceptable excipient and/or at least one additive; and
wherein the at least one oxidizing agent represents from 0.01 to 50% by weight of the oxidizing composition;
wherein the at least one oxidizing agent comprises:
a) liquid peracid and/or in-situ generated peracid; and/or
b) liquid hydrogen peroxide and/or hydrogen peroxide released from a hydrogen peroxide precursor; and
wherein the tempering composition is to be applied to the grain at a rate varying from 20 to 80 liters of the tempering composition per ton of grain.

158. A method for sanitizing mill systems, said method comprising a step of contacting the mill systems with the tempering composition defined in claim 140.

159. A method for sanitizing mill systems, said method comprising a step of contacting the mill systems with the oxidizing composition defined in claim 157.
